(19) United States
(12) Patent Application Publication
(10) Pub. No.: US 2025/0027949 A1
(43) Pub. Date: Jan. 23, 2025

(54) EVALUATING METHOD FOR RELATIVE PHARMACOLOGICAL ACTIONS OF COMBINATIONS OF IMMUNE CHECKPOINT INHIBITORS AND ANTICANCER DRUGS AS COMBINATION DRUGS COMPARED WITH PHARMACOLOGICAL ACTIONS OF IMMUNE CHECKPOINT INHIBITORS ALONE, CALCULATING METHOD, EVALUATING APPARATUS, CALCULATING APPARATUS, EVALUATING PROGRAM, CALCULATING PROGRAM, RECORDING MEDIUM, EVALUATING SYSTEM, AND TERMINAL APPARATUS

(71) Applicant: AJINOMOTO CO., INC., Tokyo (JP)

(72) Inventors: Koichi AZUMA, Fukuoka (JP); Kenta MUROTANI, Fukuoka (JP); Tetsuro SASADA, Kanagawa (JP); Tomoyuki TAGAMI, Kanagawa (JP); Asami HAGIWARA, Kanagawa (JP); Akira IMAIZUMI, Kanagawa (JP); Sachise KARAKAWA, Kanagawa (JP); Mika KAWASAKI, Kanagawa (JP); Yohei MIYAGI, Kanagawa (JP); Tomohiko TAMURA, Kanagawa (JP); Haruhiro SAITO, Kanagawa (JP); Yoshiro NAKAHARA, Kanagawa (JP); Feifei WEI, Kanagawa (JP); Rika KASAJIMA, Kanagawa (JP); Huihui XIANG, Kanagawa (JP); Tatsuma BAN, Kanagawa (JP)

(73) Assignee: AJINOMOTO CO., INC., Tokyo (JP)

(21) Appl. No.: 18/906,699

(22) Filed: Oct. 4, 2024

Related U.S. Application Data
(63) Continuation of application No. PCT/JP2023/013821, filed on Apr. 3, 2023.

(30) Foreign Application Priority Data
Apr. 8, 2022 (JP) 2022-064531

(51) Int. Cl. G01N 33/68

(52) U.S. Cl. CPC G01N 33/6812; G01N 33/6848; G01N 2800/52

(57) ABSTRACT

An evaluating method includes an evaluating step of evaluating a relative pharmacological action of a combination of an immune checkpoint inhibitor and an anticancer drug as a combination drug compared with a pharmacological action of an immune checkpoint inhibitor alone in a subject to be evaluated, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hKyn, hTrp, Kyn, KynA, NP, QA, and XA in blood of the subject to be evaluated, or (ii) a value of a formula in the absence of use of an anticancer drug as a combination drug and a value of the formula in the presence of the use that are calculated using the concentration value and the formula including an explanatory variable to be substituted with the concentration value and an explanatory variable on the presence or absence of the use.

### CROSS-REFERENCE TO RELATED APPLICATION

This application is based upon and claims the benefit of priority from PCT Application PCT/JP2023/013821, filed Apr. 3, 2023, which claims priority from Japanese Patent Application No. 2022-06:4 31, filed Apr. 8, 2022, the entire contents of which are incorporated herein by reference.

### BACKGROUND OF THE INVENTION

### 1. Field of the Invention

The present invention relates to an evaluating method for relative pharmacological actions of combinations of immune checkpoint inhibitors (ICIs) and anticancer drugs as combination drugs compared with pharmacological actions of ICIs alone, a calculating method, an evaluating apparatus, a calculating apparatus, an evaluating program, a calculating program, a recording medium, an evaluating system, and a terminal apparatus.

### 2. Description of the Related Art

Treatment selection flows that use patient background factors such as age and performance status (PS) and biomarkers such as PD-L1 protein expression in tumor tissue and tumor mutation burden (TMB) as indicators have been presented to multiple treatment regimens also including ICIs as administered drugs for non-small cell lung cancer (“Guidelines for Diagnosis and Treatment of Lung Cancer II. Non-Small Cell Lung Cancer (NSCLC) 7. Stage IV Non-Small Cell Lung Cancer (https://www.haigan.gr.jp/guideline/2020/1/2/200102070100.h tml#j_7-0_1)”).

A treatment regimen that combines ICI therapy and anticancer drug therapy has been added to the treatment flow for choosing a primary treatment for standard driver gene mutation/translocation-negative stage IV non-small cell lung cancer.

However, none of these treatment regimens benefit all patients. To the contrary, there are disadvantages such as side effects and costs. It is necessary to develop indicators for highly accurate selection of appropriate treatment for each patient. Biomarkers used in the treatment selection flows have been developed as companion diagnostics in conjunction with development of new drugs for selection of treatment with ICIs alone. The biomarkers may be used to choose anticancer drug combination therapy in ICI treatment or to choose treatment with combined immunotherapy, but there is no evidence fully established. For example, in the evaluation of PD-L1 expression in tumor tissue, ICI treatment is mainly recommended to patients with low PS level who reveal adequate PD-L1 expression level, but no biomarkers have been developed to assist in determining whether a combination of anticancer drugs should be applied in addition to ICIs. In addition to biomarkers of a type that measure target molecules, indicators for evaluating the tumor microenvironment and the host's anti-tumor immune function as well as certain types of intestinal microflora have been reported to be associated with ICI therapeutic effects, but have not yet been established as detailed, individualized indicators.

The involvement of increased energy metabolism due to active proliferation of tumor cells, catabolic states in systemic organs, and abnormal amino acid metabolism in the immune microenvironment of tumor tissue has been reported as mechanisms of amino acid metabolism change associated with the incidence of cancer (“Sikalidis A K., Amino Acids and Immune Response: A Role for Cysteine, Glutamine, Phenylalanine, Tryptophan and Arginine in T-cell Function and Cancer?, Pathol Oncol Res. 2015: 21: 9”). It has been suggested that the profiles of amino acids and related metabolites of the amino acids in blood enable evaluation of the immune microenvironment and creation of markers for predicting the efficacy of cancer immunotherapy and nutritional risk (“Hiroaki Oda, Cancer and Amino Acid Metabolism, The Journal of Biochemistry Vol. 86, No. 3, pp. 332-337 (2014)”). There have also been multiple reports on the prediction of ICI treatment prognosis using blood metabolite indices including amino acids and tryptophan metabolites (“WO 2021/090941”, “Botticelli A, Cerbelli B, Lionetto L et al., Can IDO activity predict primary resistance to anti-PD-1 treatment in NSCLC?, J Transl Med. 2018; 16(1): 219”, and “L1 H, Bullock K, Gurjao C et al., Metabolomic adaptations and correlates of survival to immune checkpoint blockade., Nat Commun. 2019; 10(1): 4346”). The correlation between multiple amino acid indices and treatment prognosis in anticancer drug therapy has also been reported (“Gey A, Tadie J M, Caumont-Prim A et al., Granulocytic myeloid-derived suppressor cells inversely correlate with plasma arginine and overall survival in critically ill patients, Clinical and Experimental Immunology, 2014; 180: 280-288”).

On the other hand, there has been no report that mentions that amino acid indices can be used to discriminate the efficacy of anticancer drug combination therapy in ICI treatment.

In short, biomarkers using amino acids or amino acid-related metabolites in blood for highly accurately discriminating or predicting the prognosis or risk of anticancer drug combination therapy in ICI treatment have not yet developed.

### SUMMARY OF THE INVENTION

It is an object of the present invention to at least partially solve the problems in the conventional technology.

The present invention was made in view of the problem. It is an object of the present invention to provide an evaluating method, a calculating method, an evaluating apparatus, a calculating apparatus, an evaluating program, a calculating program, a recording medium, an evaluating system, and a terminal apparatus that can provide highly reliable information helpful in identifying individual differences in relative pharmacological actions of combinations of ICIs and anticancer drugs as combination drugs compared with pharmacological actions of ICIs alone.

In order to solve the problem and achieve the object, an evaluating method according to one aspect of the present invention includes an evaluating step of evaluating “a relative pharmacological action of a combination of an ICI and an anticancer drug as a combination drug compared with a pharmacological action of an ICI alone” (hereinafter referred to as “relative pharmacological action”) in a subject to be evaluated, using (i) a concentration value (the concentration value may be either an absolute value or a relative value) of at least one metabolite among 21 kinds of amino acids (Glu, Arg, Orn, Cit, His, Val, Phe, Tyr, Met, Pro, Asn, Leu, Lys, Thr, Ile, Gln, Ala, Ser, a-ABA, Trp, and Gly) and 8 kinds of amino acid-related metabolites (AnthA, hKyn, hTrp, Kyn, KynA, NP, QA, and XA) in blood of the subject to be evaluated, or (ii) a value of a formula in the absence of use of an anticancer drug as a combination drug and a value of the formula in the presence of the use that are calculated using the concentration value and the formula including an explanatory variable to be substituted with the concentration value and an explanatory variable on the presence or absence of the use (hereinafter referred to as “combination presence/absence variable”).

In the present description, ICIs include PD-1 inhibitors (such as nivolumab or pembrolizumab), PD-L1 inhibitors (such as atezolizumab or durvalumab), and CTLA-4 inhibitors (such as ipilimumab). In the present description, anticancer drugs include cytotoxic anticancer drugs and molecular target drugs. In the present description, cytotoxic anticancer drugs include platinum drugs (such as carboplatin or cisplatin), antimetabolites (such as pemetrexed), topoisomerase I inhibitors, topoisomerase II inhibitors, and microtubule inhibitors (such as paclitaxel). Molecular target drugs include angiogenesis inhibitors (such as bevacizumab), anti-EGFR antibodies, EGFR inhibitors, ROS1/ALK inhibitors, ALK inhibitors, BRAF inhibitors, MEK inhibitors, and ROS1/TRK inhibitors. In the present description, pharmacological actions include drug pharmacological actions (main effects) and general pharmacological actions (adverse effects).

In the present description, various amino acids and various amino acid-related metabolites are mainly written in abbreviations, the formal names of these are as follows.

| (Abbreviation) | (Formal name) |
|---|---|
| Ala | Alanine |
| AnthA | Anthranic Acid |
| Arg | Arginine |
| Asn | Asparagine |
| ABA | α-Aminobutyric acid |
| Cit | Citrulline |
| Gln | Glutamine |
| Glu | Glutamic acid |
| Gly | Glycine |
| His | Histidine |
| hKyn | hydroxy Kynurenine |
| hTrp | 5-hydroxy Tryptophan |
| Ile | Isoleucine |
| Kyn | Kynurenine |
| KynA | Kynurenic Acid |
| Leu | Leucine |
| Lys | Lysine |
| Met | Methionine |
| NP | Neopterin |
| Orn | Ornithine |
| Phe | Phenylalanine |
| Pro | Proline |
| QA | Quinolinic Acid |
| Ser | Serine |
| Thr | Threonine |
| Trp | Tryptophan |
| Tyr | Tyrosine |
| Val | Valine |
| XA | Xanthurenic Acid |

In the evaluating method according to one aspect of the present invention, in the evaluating step, the relative pharmacological action in the subject to be evaluated is evaluated using a difference between the value of the formula in the absence of the use and the value of the formula in the presence of the use.

In the evaluating method according to one aspect of the present invention, in the evaluating step, the relative pharmacological action in the subject to be evaluated is evaluated using a combination of a result of evaluating a pharmacological action of an ICI alone in the subject to be evaluated using the value of the formula in the absence of the use and a result of evaluating a pharmacological action of the combination in the subject to be evaluated using the value of the formula in the presence of the use.

In the evaluating method according to one aspect of the present invention, the blood is taken from the subject to be evaluated after or before treatment with an ICI or treatment with an anticancer drug used as a combination drug with an ICI is started, and in the evaluating step, a relative effect (additional effect) of treatment with the combination (hereinafter referred to as “combo therapy”) compared with an effect of treatment with an ICI alone (hereinafter referred to as “monotherapy”) in the subject to be evaluated is evaluated.

In the present description, “before treatment is started” may be referred to as “before treatment” or “before the start of treatment”, and “after treatment is started” may be referred to as “after the start of treatment”. In the present description, “before the start of treatment” includes, for example, before initial treatment in a narrow sense in treatment over a certain period of time in a broad sense is performed. In the present description, “after the start of treatment” includes, for example, after initial treatment in a narrow sense in treatment over a certain period of time in a broad sense is performed and before final treatment in a narrow sense is performed (for example, generally called “during treatment”), or after final treatment in a narrow sense in treatment over a certain period of time in a broad sense is performed (for example, generally called “after treatment”).

In the evaluating method according to one aspect of the present invention, the evaluating step is performed by a control unit of an information processing apparatus including the control unit.

A calculating method according to another aspect of the present invention includes a calculating step of calculating a value of a formula for evaluating the relative pharmacological action in the absence of the use and a value of the formula in the presence of the use, using (i) a concentration value of at least one metabolite among the 21 kinds of amino acids and the 8 kinds of amino acid-related metabolites in blood of a subject to be evaluated, and (ii) the formula including an explanatory variable to be substituted with the concentration value and the combination presence/absence variable.

In the calculating method according to another aspect of the present invention, the blood is taken from the subject to be evaluated after or before treatment with an ICI or treatment with an anticancer drug used as a combination drug with an ICI is started, and the formula is to evaluate a relative effect of the combo therapy compared with an effect of the monotherapy.

In the calculating method according to another aspect of the present invention, the calculating step is performed by a control unit of an information processing apparatus including the control unit.

An evaluating apparatus according to still another aspect of the present invention is an evaluating apparatus including a control unit. The control unit includes an evaluating unit that evaluates the relative pharmacological action in a subject to be evaluated, using (i) a concentration value of at least one metabolite among the 21 kinds of amino acids and the 8 kinds of amino acid-related metabolites in blood of the subject to be evaluated, or (ii) a value of a formula in the absence of the use and a value of the formula in the presence of the use that are calculated using the concentration value and the formula including an explanatory variable to be substituted with the concentration value and the combination presence/absence variable.

In the evaluating apparatus according to still another aspect of the present invention, the evaluating apparatus is communicatively connected to a terminal apparatus via a network. The terminal apparatus provides concentration data on the concentration value or the value of the formula. The control unit further includes a data receiving unit that receives the concentration data or the value of the formula transmitted from the terminal apparatus, and a result transmitting unit that transmits an evaluation result obtained by the evaluating unit to the terminal apparatus. The evaluating unit uses the concentration value included in the concentration data or the value of the formula received by the data receiving unit.

A calculating apparatus according to still another aspect of the present invention is a calculating apparatus including a control unit. The control unit includes a calculating unit that calculates a value of a formula for evaluating the relative pharmacological action in the absence of the use and a value of the formula in the presence of the use, using (i) a concentration value of at least one metabolite among the 21 kinds of amino acids and the 8 kinds of amino acid-related metabolites in blood of a subject to be evaluated, and (ii) the formula including an explanatory variable to be substituted with the concentration value and the combination presence/absence variable.

An evaluating program according to still another aspect of the present invention is an evaluating program for causing an information processing apparatus including a control unit to perform an evaluating method. The evaluating method includes an evaluating step of evaluating the relative pharmacological action in a subject to be evaluated, using (i) a concentration value of at least one metabolite among the 21 kinds of amino acids and the 8 kinds of amino acid-related metabolites in blood of the subject to be evaluated, or (ii) a value of a formula in the absence of the use and a value of the formula in the presence of the use that are calculated using the concentration value and the formula including an explanatory variable to be substituted with the concentration value and the combination presence/absence variable.

A calculating program according to still another aspect of the present invention is a calculating program for causing an information processing apparatus including a control unit to perform a calculating method. The calculating method includes a calculating step of calculating a value of a formula for evaluating the relative pharmacological action in the absence of the use and a value of the formula in the presence of the use, using (i) a concentration value of at least one metabolite among the 21 kinds of amino acids and the 8 kinds of amino acid-related metabolites in blood of a subject to be evaluated, and (ii) the formula including an explanatory variable to be substituted with the concentration value and the combination presence/absence variable.

A recording medium according to still another aspect of the present invention is a computer readable recording medium encoded with the evaluating program or the calculating program. Specifically, the recording medium according to still another aspect of the present invention is a non-transitory tangible computer readable recording medium including programmed instructions for causing, when executed by an information processing apparatus, the information processing apparatus to perform the evaluating method or the calculating method.

An evaluating system according to still another aspect of the present invention is an evaluating system including an evaluating apparatus including a control unit and a terminal apparatus including a control unit that are communicatively connected to each other via a network. The control unit of the terminal apparatus includes (I) a data transmitting unit that transmits, to the evaluating apparatus, (i) concentration data on a concentration value of at least one metabolite among the 21 kinds of amino acids and the 8 kinds of amino acid-related metabolites in blood of a subject to be evaluated, or (ii) a value of a formula in the absence of the use and a value of the formula in the presence of the use that are calculated using the concentration value and the formula including an explanatory variable to be substituted with the concentration value and the combination presence/absence variable, and (II) a result receiving unit that receives an evaluation result transmitted from the evaluating apparatus on the relative pharmacological action. The control unit of the evaluating apparatus includes (I) a data receiving unit that receives the concentration data or the value of the formula transmitted from the terminal apparatus, (II) an evaluating unit that evaluates the relative pharmacological action in the subject to be evaluated, using the concentration value included in the concentration data or the value of the formula received by the data receiving unit, and (III) a result transmitting unit that transmits the evaluation result obtained by the evaluating unit to the terminal apparatus.

A terminal apparatus according to still another aspect of the present invention is a terminal apparatus including a control unit. The control unit includes a result obtaining unit that obtains an evaluation result on a relative pharmacological action. The evaluation result is a result of evaluating the relative pharmacological action in a subject to be evaluated, using (i) a concentration value of at least one metabolite among the 21 kinds of amino acids and the 8 kinds of amino acid-related metabolites in blood of the subject to be evaluated, or (ii) a value of a formula in the absence of the use and a value of the formula in the presence of the use that are calculated using the concentration value and the formula including an explanatory variable to be substituted with the concentration value and the combination presence/absence variable.

The terminal apparatus according to still another aspect of the present invention is communicatively connected via a network to an evaluating apparatus that evaluates the relative pharmacological action. The control unit includes a data transmitting unit that transmits concentration data on the concentration value or the value of the formula to the evaluating apparatus. The result obtaining unit receives the evaluation result transmitted from the evaluating apparatus.

The present invention achieves the effect of providing highly reliable information helpful in identifying individual differences in the relative pharmacological actions.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a principle configurational diagram showing a basic principle of a first embodiment;

FIG. 2 is a principle configurational diagram showing a basic principle of a second embodiment;

FIG. 3 is a diagram showing an example of an entire configuration of a present system;

FIG. 4 is a diagram showing another example of an entire configuration of the present system;

FIG. 5 is a block diagram showing an example of a configuration of an evaluating apparatus 100 in the present system;

FIG. 6 is a chart showing an example of information stored in a concentration data file 106a;

FIG. 7 is a chart showing an example of information stored in an index state information file 106b;

FIG. 8 is a chart showing an example of information stored in a designated index state information file 106c;

FIG. 9 is a chart showing an example of information stored in a formula file 106d1;

FIG. 10 is a chart showing an example of information stored in an evaluation result file 106e;

FIG. 11 is a block diagram showing a configuration of an evaluating part 102d;

FIG. 12 is a block diagram showing an example of a configuration of a client apparatus 200 in the present system;

FIG. 13 is a block diagram showing an example of a configuration of a database apparatus 400 in the present system;

FIG. 14 is a table showing the results of correlation analysis between the measurement values of amino acids and amino acid-related metabolites in plasma before the start of treatment and the prognosis of treatment (OS);

FIG. 15 is a table showing the results of correlation analysis between the measurement values of amino acids and amino acid-related metabolites in plasma before the start of treatment and the prognosis of treatment (OS);

FIG. 16A is a table showing information on multivariate discriminants based on a covariate model;

FIG. 16B is a table showing information on multivariate discriminants based on a covariate model;

FIG. 17A is a table showing information on multivariate discriminants based on a stratified model;

FIG. 17B is a table showing information on multivariate discriminants based on a stratified model;

FIG. 18 is a chart showing a distribution of risk scores;

FIG. 19 is a chart showing survival time curves;

FIG. 20 is a chart showing a distribution of risk scores; and

FIG. 21 is a chart showing survival time curves.

### DETAILED DESCRIPTION OF THE PREFERRED EMBODIMENTS

An embodiment (first embodiment) of an evaluating method according to the present invention and an embodiment (second embodiment) of an evaluating apparatus, an evaluating method, an evaluating program, a recording medium, an evaluating system, and a terminal apparatus according to the present invention will be explained in detail below with reference to the drawings. The present invention is not limited by these embodiments.

### First Embodiment

### 1-1. Overview of First Embodiment

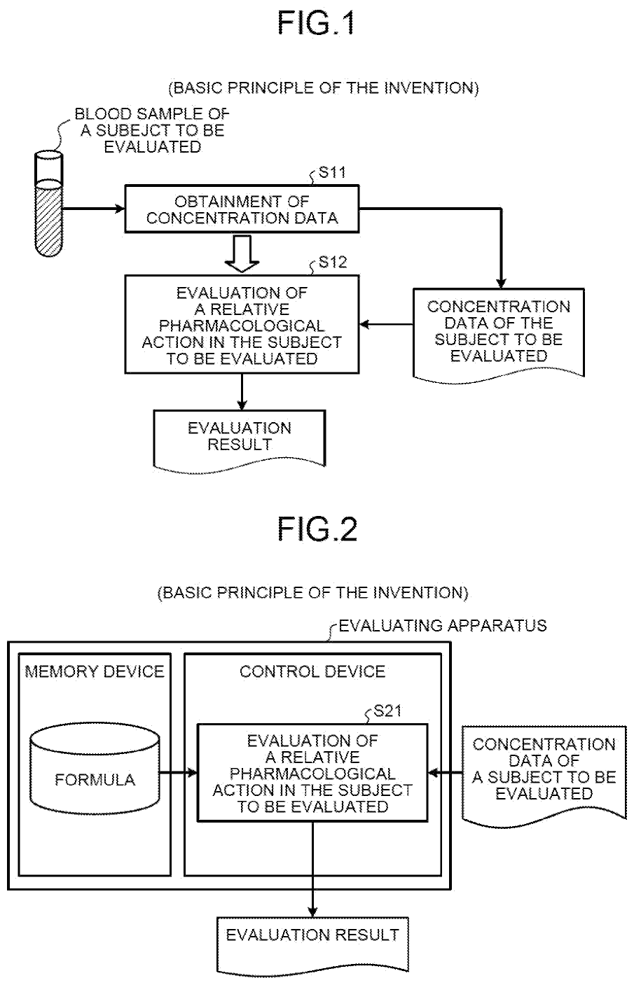

An overview of the first embodiment will be explained with reference to FIG. 1. FIG. 1 is a principle configuration diagram showing a basic principle according to the first embodiment.

Concentration data on a concentration value of at least one metabolite among the 21 kinds of amino acids and the 8 kinds of amino acid-related metabolites in blood (for example, including plasma and serum) taken from a subject with cancer to be evaluated (for example, an individual such as an animal or a human) that may receive monotherapy or combo therapy is obtained (step S11).

As used herein “may receive monotherapy or combo therapy” means, for example, that monotherapy or combo therapy may be chosen or monotherapy or combo therapy is scheduled.

At step S11, for example, one or both of concentration data derived from blood taken from the subject to be evaluated before cancer treatment (for example, treatment by surgical therapy, chemotherapy, radiotherapy, or cancer immunotherapy) is started (concentration data before the start of treatment) and concentration data derived from blood taken after the treatment is started (concentration data after the start of treatment) may be obtained. The term “before the start of treatment” includes, for example, before initial treatment in a narrow sense in treatment over a certain period of time in a broad sense is performed. The term “after the start of treatment” includes, for example, after initial treatment in a narrow sense in treatment over a certain period of time in a broad sense is performed and before final treatment in a narrow sense is performed (for example, generally called “during treatment”), or after final treatment in a narrow sense in treatment over a certain period of time in a broad sense is performed (for example, generally called “after treatment”).

At step S11, for example, the concentration data measured by a company or the like that measures concentrations may be obtained. For example, the following measuring method of (A), (B), or (C) may be used to measure concentrations from the blood extracted from the subject to be evaluated to obtain the concentration data. In the present description, the unit of the concentration may be, for example, molar concentration, weight concentration, enzyme activity, or one obtained by addition, subtraction, multiplication, and division of any constant with these concentrations. The concentration may be any of an absolute value or a relative value.

(A) Plasma is separated from blood by centrifuging the collected blood sample. All plasma samples are frozen and stored at −80° C. until the concentration is measured. At the time of measuring the concentration, after acetonitrile is added to deproteinize the plasma samples, impurities such as phospholipid are removed by solid phase extraction as needed, pre-column derivatization is then performed using a labeling reagent (3-aminopyridyl-N-hydroxysuccinimidyl carbamate), and the concentration is analyzed by liquid chromatograph mass spectrometry (LC/MS) (see International Publication WO 2003/069328, International Publication WO 2005/116629, or Nonpatent Literature “Chromatography 2019, 40, 127-133”).
(B) Plasma is separated from blood by centrifuging the collected blood sample. All plasma samples are frozen and stored at −80° C. until the concentration is measured. At the time of measuring the concentration, sulfosalicylic acid is added to deproteinize the plasma samples, and the concentration is analyzed by an amino acid analyzer based on post-column derivatization using a ninhydrin reagent.
(C) Blood cell separation is performed on the collected blood sample by using a membrane, micro-electro-mechanical System (MEMS) technology, or the principle of centrifugation, whereby plasma or serum is separated from the blood. A plasma or serum sample the concentration of which is not measured immediately after obtaining the plasma or the serum is frozen and stored at −80° C. until the concentration is measured. At the time of measuring the concentration, a molecule that reacts with or binds to a target substance in blood, such as an enzyme, an aptamer, or an antibody, is used to perform quantitative analysis and the like on an increasing or decreasing substance or a spectroscopic value by substrate recognition, whereby the concentration is analyzed.

Subsequently, a relative pharmacological action in the subject to be evaluated is evaluated (predicted) using the concentration value included in the concentration data obtained at step S11 (step S12). Before step S12 is performed, data such as defectives and outliers may be removed from the concentration data obtained at step S11. As used herein “a relative pharmacological action in the subject to be evaluated is evaluated” means, for example, that a relative pharmacological action developed in the subject to be evaluated is evaluated. When both of the concentration data before the start of treatment and the concentration data after the start of treatment are used at step S12, for example, the ratio or the difference between the concentration value before the start of treatment and the concentration value after the start of treatment may be calculated, and the calculated ratio or difference may be used to perform evaluation. At step S12, the relative effect (prognosis of treatment) of combo therapy compared with the effect of monotherapy (prognosis of treatment), that is, the additional effect in the subject to be evaluated may be evaluated using the concentration value included in any one or both of the concentration data before the start of treatment and the concentration data after the start of treatment.

As described above, in the first embodiment, at step S11, the concentration data of the subject to be evaluated is obtained, and at step S12, the relative pharmacological action in the subject to be evaluated is evaluated using the concentration value included in the concentration data of the subject to be evaluated obtained at step S11 (in short, information for evaluating the relative pharmacological action in the subject to be evaluated is obtained). This method can provide highly reliable information helpful in identifying individual differences in relative pharmacological action. In particular, when only the concentration data before the start of treatment is used at step S12, the evaluation result obtained in the present embodiment can be utilized as reference information for deciding a treatment method. When the concentration data after the start of treatment or after treatment is used at step S12, the evaluation result obtained in the present embodiment can be utilized to determine whether to continue treatment or can be utilized as reference information for deciding another treatment method.

It may be determined that the concentration value (which may be the ratio or the difference) included in the concentration data obtained at step S11 reflects the relative pharmacological action in the subject to be evaluated. The concentration value (which may be the ratio or the difference) may be converted using, for example, the methods listed below, and it may be determined that the converted value reflects the relative pharmacological action in the subject to be evaluated. In other words, the concentration value or the converted value itself may be treated as the evaluation result on the relative pharmacological action in the subject to be evaluated.

The concentration value may be converted such that a possible range of the concentration value falls within a predetermined range (for example, the range from 0.0 to 1.0, the range from 0.0 to 10.0, the range from 0.0 to 100.0, or the range from −10.0 to 10.0), for example, by addition, subtraction, multiplication, and division of any given value with the concentration value, by conversion of the concentration value by a predetermined conversion method (for example, exponential transformation, logarithm transformation, angular transformation, square root transformation, probit transformation, reciprocal transformation, Box-Cox transformation, or power transformation), or by performing a combination of these computations on the concentration value. For example, a value of an exponential function with the concentration value as an exponent and Napier constant as the base (specifically, a value of p/(1−p) where a natural logarithm ln(p/(1−p)) is equal to the concentration value when the probability p that the prognosis of treatment is not good is defined) may be further calculated, and a value (specifically, a value of the probability p) may be further calculated by dividing the calculated value of the exponential function by the sum of 1 and the value of the exponential function.

The concentration value may be converted such that the converted value is a particular value when a particular condition is met. For example, the concentration value may be converted such that the converted value is 5.0 when the specificity is 80% and the converted value is 8.0 when the specificity is 95%.

After normally distributing the concentration distribution for each amino acid and for each amino acid-related metabolite, the concentration distribution may be standardized such that the mean is 50 and the standard deviation is 10.

These conversions may be performed by gender or age.

The concentration value in the present description may be the concentration value itself or may be the converted value of the concentration value.

Positional information about a position of a predetermined mark on a predetermined scale visually presented on a display device such as a monitor or a physical medium such as paper may be generated using the concentration value (which may be the ratio or the difference) included in the concentration data obtained at step S11 or, if the concentration value is converted, the converted value, and it may be determined that the generated positional information reflects the relative pharmacological action in the subject to be evaluated. The predetermined scale is for evaluating the relative pharmacological action in the subject to be evaluated and is, for example, a graduated scale at least marked with graduations corresponding to the upper limit value and the lower limit value in “a possible range of the concentration value or the converted value” or “part of the range”. The predetermined mark corresponds to the concentration value or the converted value and is, for example, a circle sign or a star sign.

If the concentration value (which may be the ratio or the difference) included in the concentration data obtained at step S11 is lower than a predetermined value (e.g., mean±1SD, 2SD, 3SD, N quantile, N percentile, or a cutoff value the clinical significance of which is recognized) or is equal to or lower than the predetermined value, or the concentration value is equal to or higher than the predetermined value or is higher than the predetermined value, the relative pharmacological action in the subject to be evaluated may be evaluated. In this case, instead of the concentration value itself, a concentration standard score (a value obtained by normally distributing the concentration distribution by gender and then standardizing the concentration value with a mean of 50 and a standard deviation of 10 for each amino acid and each amino acid-related metabolite) may be used. For example, if the concentration standard score is lower than the mean −2SD (when the concentration standard score <30) or if the concentration standard score is higher than the mean+2SD (when the concentration standard score >70), the relative pharmacological action in the subject to be evaluated may be evaluated.

The relative pharmacological action in the subject to be evaluated may be evaluated by calculating a value of a formula in the absence of use of an anticancer drug as a combination drug and a value of the formula in the presence of the use, using the concentration value (which may be the ratio or the difference) and the formula including an explanatory variable to be substituted with the concentration value (which may be the ratio or the difference) included in the concentration data obtained at step S11 and the combination presence/absence variable. For example, the relative pharmacological action in the subject to be evaluated may be evaluated using a difference between the value of the formula in the absence of the use and the value of the formula in the presence of the use. For example, the pharmacological action of an ICI alone in the subject to be evaluated may be evaluated using the value of the formula in the absence of the use, the pharmacological action of the combination of an ICI and an anticancer drug as a combination drug in the subject to be evaluated may be evaluated using the value of the formula in the presence of the use, and the relative pharmacological action in the subject to be evaluated may be evaluated using these obtained evaluation results.

It may be determined that the calculated value of the formula reflects the relative pharmacological action in the subject to be evaluated. The value of the formula may be converted using, for example, the methods listed below, and it may be determined that the converted value reflects the relative pharmacological action in the subject to be evaluated. In other words, the value of the formula or the converted value itself may be treated as the evaluation result on the relative pharmacological action in the subject to be evaluated.

The value of the formula may be converted such that a possible range of the value of the formula falls within a predetermined range (for example, the range from 0.0 to 1.0, the range from 0.0 to 10.0, the range from 0.0 to 100.0, or the range from −10.0 to 10.0), for example, by addition, subtraction, multiplication, and division of any given value with the value of the formula, by conversion of the value of the formula by a predetermined conversion method (for example, exponential transformation, logarithm transformation, angular transformation, square root transformation, probit transformation, reciprocal transformation, Box-Cox transformation, or power transformation), or by performing a combination of these computations on the value of the formula. For example, a value of an exponential function with the value of the formula as an exponent and Napier constant as the base (specifically, a value of p/(1−p) where a natural logarithm ln(p/(1−p)) is equal to the value of the formula when the probability p that the prognosis of treatment is not good is defined) may be further calculated, and a value (specifically, a value of the probability p) may be further calculated by dividing the calculated value of the exponential function by the sum of 1 and the value of the exponential function.

The value of the formula may be converted such that the converted value is a particular value when a particular condition is met. For example, the value of the formula may be converted such that the converted value is 5.0 when the specificity is 80% and the converted value is 8.0 when the specificity is 95%.

The value of the formula may be standardized such that the mean is 50 and the standard deviation is 10.

These conversions may be performed by gender or age.

The value of the formula in the present description may be the value of the formula itself or may be the converted value of the value of the formula.

Positional information about a position of a predetermined mark on a predetermined scale visually presented on a display device such as a monitor or a physical medium such as paper may be generated using the value of the formula or, if the value of the formula is converted, the converted value, and it may be determined that the generated positional information reflects the relative pharmacological action in the subject to be evaluated. The predetermined scale is for evaluating the relative pharmacological action in the subject to be evaluated and is, for example, a graduated scale at least marked with graduations corresponding to the upper limit value and the lower limit value in “a possible range of the value of the formula or the converted value” or “part of the range”. The predetermined mark corresponds to the value of the formula or the converted value and is, for example, a circle sign or a star sign.

The relative pharmacological action in the subject to be evaluated may be qualitatively evaluated. Specifically, the subject to be evaluated may be classified into any one of a plurality of categories defined at least in consideration of the relative prognosis of treatment with combo therapy compared with the prognosis of treatment with monotherapy, using “the concentration value (which may be the ratio or the difference) included in the concentration data obtained at step S11 and preset one or more thresholds” or “the concentration value (which may be the ratio or the difference) included in the concentration data, a formula including an explanatory variable to be substituted with the concentration value (which may be the ratio or the difference) and the combination presence/absence variable, and preset one or more thresholds”. The categories may include a category to which a subject with poor prognosis of treatment belongs, a category to which a subject with good prognosis of treatment belongs, and a category to which a subject with prognosis of treatment being intermediate between good and poor belongs. The categories may include a category to which a subject with poor prognosis of treatment belongs and a category to which a subject with good prognosis of treatment belongs. The concentration value (which may be the ratio or the difference) or the value of the formula may be converted by a predetermined method, and the subject to be evaluated may be classified into any one of the categories using the converted value.

As for the formula used for the evaluation, the form of the formula is not specifically designated, however, for example, may be the following forms.
linear model such as multiple regression equation, linear discriminant, principal component analysis, and canonical discriminant analysis that are based on the least-squares method;
generalized linear model such as logistic regression and Cox regression that are based on the maximum likelihood method;
generalized linear mixed model considering random effects due to individual differences, facility differences, and other factors in addition to the generalized linear model
expression generated by cluster analysis such as the K-means method and hierarchical cluster analysis;
expression generated on the basis of the Bayesian statistics such as the Markov chain Monte Carlo (MCMC), the Bayesian network, and the hierarchical Bayesian method;
expression generated by class classification such as support vector machine and decision tree;
expression generated by a method that does not belong to the above-cited categories such as fractional expression; and
expression represented as, for example, the summation of expressions of different forms

The formula used for the evaluation may be prepared by a method described in WO 2004/052191 that is an international application filed by the present applicant or by a method described in WO 2006/098192 that is an international application filed by the present applicant. Any formulae obtained by these methods can be preferably used in the evaluation of the relative pharmacological action, regardless of the units of concentrations of amino acids or amino acid-related metabolites in the concentration data as input data.

In the multiple regression equation, the multiple logistic regression equation, and the canonical discriminant function, a coefficient and a constant term are added to each explanatory variable, and the coefficient and the constant term may be preferably real numbers, more preferably values in the range of 99% confidence interval for the coefficient and the constant term obtained from data for the various kinds of classifications described above, more preferably values in the range of 95% confidence interval for the coefficient and the constant term obtained from data for the various kinds of classifications described above. The value of each coefficient and the confidence interval thereof may be those multiplied by a real number, and the value of the constant term and the confidence interval thereof may be those having an arbitrary actual constant added or subtracted or those multiplied or divided by an arbitrary actual constant. When an expression such as the logistic regression, the linear discriminant, and the multiple regression equation is used for the evaluation, a linear transformation of the expression (addition of a constant and multiplication by a constant) and a monotonic increasing (decreasing) transformation (for example, a logit transformation) of the expression do not alter evaluation performance and thus evaluation performance after transformation is equivalent to that before transformation. Therefore, the expression includes an expression that is subjected to the linear transformation and the monotonic increasing (decreasing) transformation.

In the fractional expression, the numerator of the fractional expression is expressed by the sum of the explanatory variables A, B, C etc. and the denominator of the fractional expression is expressed by the sum of the explanatory variables a, b, c etc. The fractional expression also includes the sum of the fractional expressions α, β, γ etc. (for example, α+β) having such constitution. The fractional expression also includes divided fractional expressions. The explanatory variables used in the numerator or denominator may have suitable coefficients respectively. The explanatory variables used in the numerator or denominator may appear repeatedly. Each fractional expression may have a suitable coefficient. A value of a coefficient for each explanatory variable and a value for a constant term may be any real numbers. In a fractional expression and the one in which explanatory variables in the numerator and explanatory variables in the denominator in the fractional expression are switched with each other, the positive and negative signs are generally reversed in correlation with objective explanatory variables, but because their correlation is maintained, the evaluation performance can be assumed to be equivalent. The fractional expression therefore also includes the one in which explanatory variables in the numerator and explanatory variables in the denominator in the fractional expression are switched with each other.

When the relative pharmacological action is evaluated, in addition to the concentration value of at least one metabolite among the 21 kinds of amino acids and the 8 kinds of amino acid-related metabolites, values on other biological information (for example, values listed below) may be further used. The formula to be used in evaluation may further include one or more explanatory variables to be substituted with the values on other biological states (for example, values listed below), in addition to the explanatory variable to be substituted with the concentration value.

1. Concentration values of metabolites (carbohydrates, lipids, etc.) in blood, other than amino acids and amino acid-related metabolites, proteins, peptides, minerals, hormones, and the like
2. Blood test values such as tumor markers, albumin, total protein, triglycerides, HbA1c, LDL cholesterol, HDL cholesterol, amylase, total bilirubin, and uric acid
3. Immune-related test values such as blood cytokines, immunocompetent cell count, cytokines in immunocompetent cells, and delayed type hyperreactivity (DTH)
4. Values obtained from image information from ultrasound echo, upper and lower endoscopy, X-ray, CT, MRI, and the like
5. Values related to biological indicators such as age, height, weight, BMI, blood pressure, gender, smoking information, diet information, drinking information, exercise information, stress information, sleep information, family medical history information, and disease history information (diabetes, pancreatitis, etc.)
6. Values obtained from multilayer omics analysis information, information on cancer gene mutations, information on microsatellite instability, information on cancer-derived antigens and antibodies, or information on the expression of molecules such as PD-1 and PD-L1

### Second Embodiment

### 2-1. Outline of the Second Embodiment

Here, outlines of the second embodiment will be described in detail with reference to FIG. 2. FIG. 2 is a principle configurational diagram showing a basic principle of the second embodiment. In the description of the present second embodiment, description duplicating that of the first embodiment is sometimes omitted. In particular, herein, when the relative pharmacological action is evaluated, a case of using the value of the formula or the converted value thereof is described as one example. However, for example, the concentration value, the rario or the dirrerence of the concentration values, or the converted value thereof (for example, a concentration standard score) may be used.

A control device evaluates the relative pharmacological action in the subject to be evaluated by calculating the value of the formula using (i) the concentration value included the previously obtained concentration data on the concentration value of at least one metabolite among the 21 kinds of amino acids and the 8 kinds of amino acid-related metabolites in blood taken from the subject with cancer to be evaluated (for example, an individual such as an animal or a human) that may receive monotherapy or combo therapy and (ii) the formula previously stored in a memory device, including the explanatory variable to be substituted with the concentration value and the combination presence/absence variable (step S21). When both of the concentration data before the start of treatment and the concentration data after the start of treatment are used at step S21, for example, the control device may evaluate the relative pharmacological action in the subject to be evaluated by calculating the ratio or the difference between the concentration value before the start of treatment and the concentration value after the start of treatment and calculating the value of the formula by substituting the calculated ratio or difference for the explanatory variable. This apparatus can provide highly reliable information helpful in identifying individual differences in the relative pharmacological actions.

The formula used at step S21 may be generated based on the formula-preparing processing (step 1 to step 4) described below. Here, the summary of the formula-preparing processing is described. The processing described below is merely one example, and the method of preparing the formula is not limited thereto.

First, the control device prepares a candidate formula (e.g., y=a1x1+a2x2+ . . . +anxn, y: index data, xi: concentration data or combination presence/absence data, ai: constant, i=1, 2, . . . , n) based on a predetermined formula-preparing method from index state information (data such as defectives and outliers may be removed in advance) previously stored in the memory device (step 1). The index state information includes concentration data of a patient (for example, concentration data of amino acids and amino acid-related metabolites before the start of treatment, concentration data of amino acids and amino acid-related metabolites after the start of treatment, or concentration data on the amount of change of amino acids and amino acid-related metabolites between before the start of treatment and after the start of treatment), combination presence/absence data on the presence or absence of the use of an anticancer drug as a combination drug, and index data of the patient on the prognosis of treatment (for example, binary data on poor or good prognosis of treatment).

In step 1, a plurality of the candidate formulae may be prepared from the index state information by using a plurality of the different formula-preparing methods (including those for multivariate analysis such as the principal component analysis, the discriminant analysis, the support vector machine, the multiple regression analysis, the Cox regression analysis, the logistic regression analysis, the K-means method, the cluster analysis, and the decision tree). Specifically, a plurality of groups of the candidate formulae may be prepared simultaneously and concurrently by using a plurality of different algorithms with the index state information which is multivariate data including the concentration data obtained by analyzing any one or both of blood taken before treatment and blood taken after the start of treatment from a large number of patients, the combination presence/absence data obtained from the patients, and the index data. For example, the two different candidate formulae may be formed by performing the discriminant analysis and the logistic regression analysis simultaneously with the different algorithms. Alternatively, the candidate formula may be formed by converting the index state information with the candidate formula prepared by performing the principal component analysis and then performing the discriminant analysis of the converted index state information. In this way, it is possible to finally prepare the most appropriate formula for the evaluation.

The candidate formula prepared by the principal component analysis is a linear expression including each explanatory variable maximizing the variance of all concentration data. The candidate formula prepared by the discriminant analysis is a high-powered expression (including exponential and logarithmic expressions) including each explanatory variable minimizing the ratio of the sum of the variances in respective groups to the variance of all concentration data. The candidate formula prepared by using the support vector machine is a high-powered expression (including kernel function) including each explanatory variable maximizing the boundary between groups. The candidate formula prepared by using the multiple regression analysis is a high-powered expression including each explanatory variable minimizing the sum of the distances from all concentration data. The candidate formula prepared by using the Cox regression analysis is a linear model including a logarithmic hazard ratio, and is a linear expression including each explanatory variable with a coefficient thereof maximizing the likelihood of the linear model. The candidate formula prepared by using the logistic regression analysis is a linear model expressing logarithmic odds of probability, and a linear expression including each explanatory variable maximizing the likelihood of the probability. The K-means method is a method of searching k pieces of neighboring concentration data in various groups, designating the group containing the greatest number of the neighboring points as its data-belonging group, and selecting the explanatory variable that makes the group to which input concentration data belong agree well with the designated group. The cluster analysis is a method of clustering (grouping) the points closest in entire concentration data. The decision tree is a method of ordering explanatory variables and predicting the group of concentration data from the pattern possibly held by the higher-ordered explanatory variable.

Returning to the description of the formula-preparing processing, the control device verifies (mutually verifies) the candidate formula prepared in step 1 based on a particular verifying method (step 2). The verification of the candidate formula is performed on each other to each candidate formula prepared in step 1. In step 2, at least one of discrimination rate, sensitivity, specificity, information criterion (Akaike information criterion (AIC), Bayesian information criterion (BIC)), ROC_AUC (area under the curve in a receiver operating characteristic curve), C-index (Concordance index), and the like of the candidate formula may be verified by at least one of bootstrap method, holdout method, N-fold method, leave-one-out method, and the like. In this way, it is possible to prepare the candidate formula higher in predictability or reliability, by taking the index state information and the evaluation condition into consideration.

The discrimination rate is a rate in which a subject to be evaluated whose true state is negative (for example, a subject with good prognosis of the treatment) is correctly evaluated as being negative by the evaluation method according to the present embodiment and a subject to be evaluated whose true state is positive (for example, a subject with poor prognosis of the treatment) is correctly evaluated as being positive by the evaluation method according to the present embodiment. The sensitivity is a rate in which a subject to be evaluated whose true state is positive is correctly evaluated as being positive by the evaluation method according to the present embodiment. The specificity is a rate in which a subject to be evaluated whose true state is negative is correctly evaluated as being negative by the evaluation method according to the present embodiment. The Akaike information criterion (AIC) is a criterion representing how observation data agrees with a statistical model, for example, in the regression analysis, and it is determined that the model in which the value defined by “−2×(maximum log-likelihood of statistical model)+2×(the number of free parameters of statistical model)” is smallest is the best. The Bayesian information criterion (BIC) is a model selection criterion derived based on the concept of Bayesian statistics. A model with the smallest value defined by “−2×(maximum log-likelihood of statistical model)+(the number of free parameters of statistical model)×ln (sample size)” (a model with a fewer parameters) is determined to be the best. ROC_AUC is defined as the area under the receiver operating characteristics curve (ROC) created by plotting (x, y)=(1−specificity, sensitivity) on two-dimensional coordinates. The value of ROC_AUC is 1 in perfect discrimination, and the closer this value is to 1, the higher the discriminative characteristic. The C-index is an index of the accuracy of prognosis prediction proposed by Harrell et al. and is a non-parametric index that indicates how much the magnitudes of the probability of event occurrence predicted by a model and the probability of the actual event occurrence match. The predictability is the average of discrimination rates, sensitivities, or specificities obtained by repeating the validation of the candidate formula. The robustness refers to the variance of discrimination rates, sensitivities, or specificities obtained by repeating the validation of the candidate formula.

Returning to the description of the formula-preparing processing, the control device selects a combination of the concentration data contained in the index state information used in preparing the candidate formula, by selecting an explanatory variable of the candidate formula based on a predetermined explanatory variable-selecting method (step 3). In step 3, the selection of the explanatory variable may be performed on each candidate formula prepared in step 1. In this way, it is possible to select the explanatory variable of the candidate formula properly. The step 1 is executed once again by using the index state information including the concentration data selected in step 3. In step 3, the explanatory variable of the candidate formula may be selected based on at least one of stepwise method, best path method, local search method, and genetic algorithm from the verification result obtained in step 2. The best path method is a method of selecting an explanatory variable by optimizing an evaluation index of the candidate formula while eliminating the explanatory variables contained in the candidate formula one by one.

Returning to the description of the formula-preparing processing, the control device prepares the formula used for the evaluation by repeatedly performing steps 1, 2 and 3, and based on the verification results thus accumulated, selecting the candidate formula used for the evaluation from the candidate formulae (step 4). In the selection of the candidate formula, there are cases where the optimum formula is selected from the candidate formulae prepared in the same formula-preparing method or the optimum formula is selected from all candidate formulae.

As explained above, in the formula-preparing processing, the processing for the preparation of the candidate formulae, the verification of the candidate formulae, and the selection of the explanatory variables in the candidate formulae are performed based on the index state information in a series of operations in a systematized manner, whereby the formula most appropriate for evaluating the relative pharmacological action can be prepared. In other words, in the formula-preparing processing, the concentration values of amino acids and amino acid-related metabolites and the presence or absence of the combination with an anticancer drug are used in multivariate statistical analysis, and for selecting the optimum and robust combination of the explanatory variables, the explanatory variable-selecting method is combined with cross-validation to extract the formula having high evaluation performance.

### 2-2. System Configuration

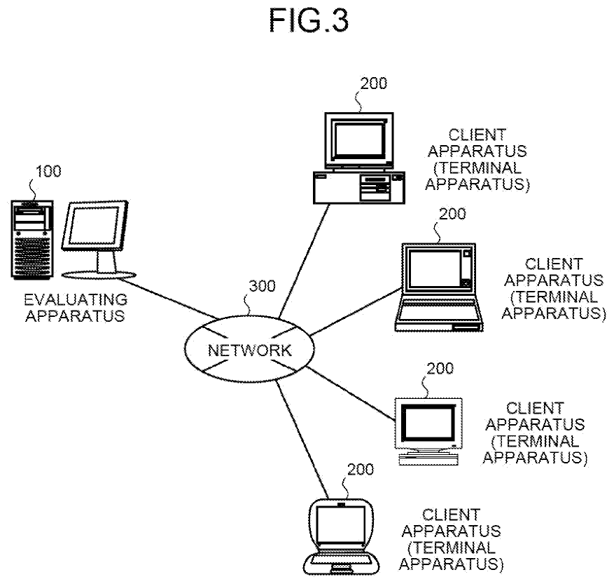

Hereinafter, the configuration of the evaluating system according to the second embodiment (hereinafter referred to sometimes as the present system) will be described with reference to FIGS. 3 to 13. This system is merely one example, and the present invention is not limited thereto. In particular, herein, when the relative pharmacological action is evaluated, a case of using the value of the formula or the converted value thereof is described as one example. However, for example, the concentration value, the rario or the dirrerence of the concentration values, or the converted value thereof (for example, a concentration standard score) may be used.

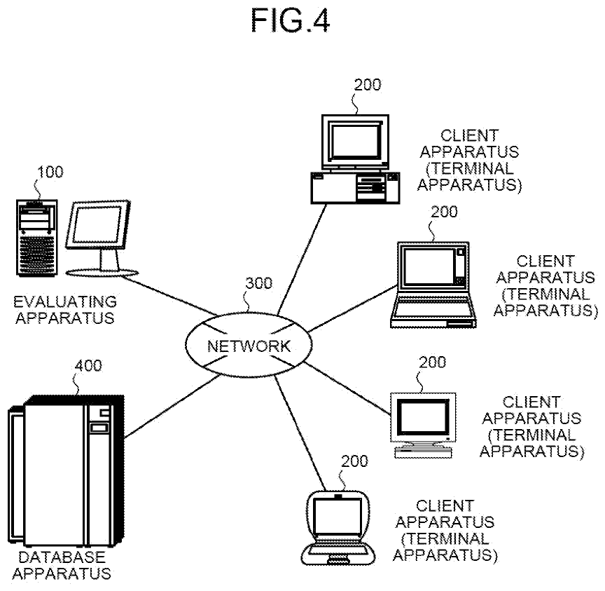

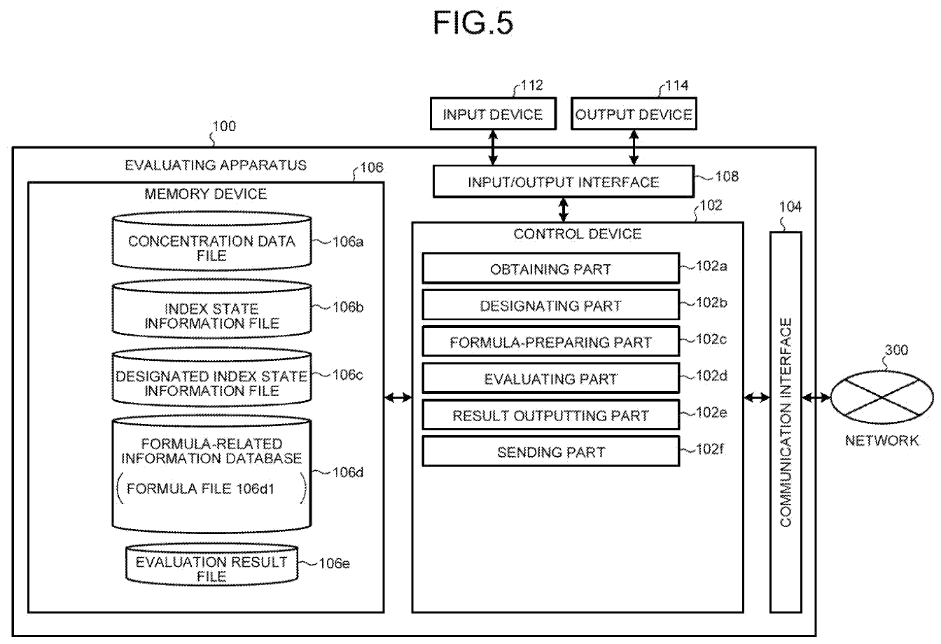

First, an entire configuration of the present system will be described with reference to FIGS. 3 and 4. FIG. 3 is a diagram showing an example of the entire configuration of the present system. FIG. 4 is a diagram showing another example of the entire configuration of the present system. As shown in FIG. 3, the present system is constituted in which the evaluating apparatus 100 that evaluates the relative pharmacological action in the individual as the subject to be evaluated and the client apparatus 200 (corresponding to the terminal apparatus of the present invention) that provides the concentration data of the individual, are communicatively connected to each other via a network 300.

In the present system, the client apparatus 200 that provides data for use in the evaluation and the client apparatus 200 that receives the evaluation result may be different. In the present system as shown in FIG. 4, in addition to the evaluating apparatus 100 and the client apparatus 200, the database apparatus 400 storing, for example, the index state information used in preparing the formula in the evaluating apparatus 100 and the formula used for the evaluation, may be communicatively connected via the network 300.

Now, the configuration of the evaluating apparatus 100 in the present system will be described with reference to FIGS. 5 to 11. FIG. 5 is a block diagram showing an example of the configuration of the evaluating apparatus 100 in the present system, showing conceptually only the region relevant to the present invention.

The evaluating apparatus 100 includes: (i) a control device 102, such as CPU (Central Processing Unit), that integrally controls the evaluating apparatus; (ii) a communication interface 104 that connects the evaluating apparatus to the network 300 communicatively via communication apparatuses such as a router and wired or wireless communication lines such as a private line; (iii) a memory device 106 that stores various databases, tables, files and others; and (iv) an input/output interface 108 connected to an input device 112 and an output device 114, and these parts are connected to each other communicatively via any communication channel. The evaluating apparatus 100 may be present together with various analyzers (e.g., an analyzer for amino acids and amino acid-related metabolites) in a same housing. For example, the evaluating apparatus 100 may be a compact analyzing device including components (hardware and software) that calculate (measure) the concentration value of at least one metabolite among the 21 kinds of amino acids and the 8 kinds of amino acid-related metabolites in blood and output (e.g., print or display on a monitor) the calculated value, wherein the compact analyzing device is characterized by further including the evaluating part 102d described later, and using the components to output results obtained by the evaluating part 102d.

The communication interface 104 allows communication between the evaluating apparatus 100 and the network 300 (or a communication apparatus such as a router). Thus, the communication interface 104 has a function to communicate data via a communication line with other terminals.

The input/output interface 108 is connected to the input device 112 and the output device 114. A monitor (including a home television), a speaker, or a printer may be used as the output device 114 (hereinafter, the output device 114 may be described as the monitor 114). A keyboard, a mouse, a microphone, or a monitor functioning as a pointing device together with a mouse may be used as the input device 112.

The memory device 106 is a storage means, and examples thereof include a memory apparatus such as RAM (Random Access Memory) and ROM (Read Only Memory), a fixed disk drive such as a hard disk, a flexible disk, and an optical disk. The memory device 106 stores computer programs giving instructions to the CPU for various processings, together with OS (Operating System). As shown in the figure, the memory device 106 stores the concentration data file 106a, the index state information file 106b, the designated index state information file 106c, a formula-related information database 106d, and the evaluation result file 106e.

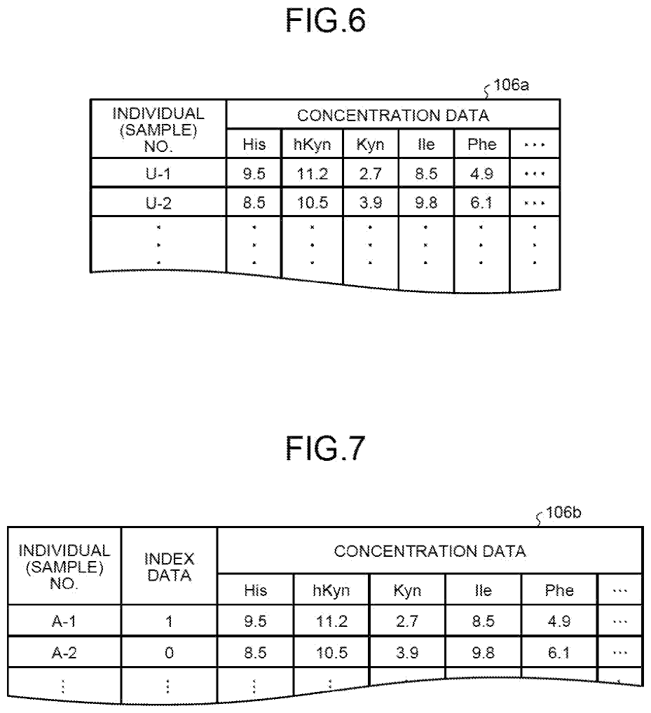

The concentration data file 106a stores the concentration data (for example, any one or both of the concentration data before the start of treatment and the concentration data after the start of treatment). FIG. 6 is a chart showing an example of information stored in the concentration data file 106a. As shown in FIG. 6, the information stored in the concentration data file 106a includes an individual number for uniquely identifying the individual (sample) as the subject to be evaluated and the concentration data that are correlated to one another. In FIG. 6, the concentration data is assumed to be numerical values, i.e., on a continuous scale, but the concentration data may be expressed on a nominal scale or an ordinal scale. In the case of the nominal or ordinal scale, any number may be allocated to each state for analysis. The concentration data may be combined with a value related to the other biological information (see above).

Returning to FIG. 5, the index state information file 106b stores the index state information used in preparing the formula. FIG. 7 is a chart showing an example of information stored in the index state information file 106b. As shown in FIG. 7, the information stored in the index state information file 106b includes the individual number, the index data, and the concentration data that are correlated to one another. In FIG. 7, the index data and the concentration data are assumed to be numerical values, i.e., on a continuous scale, but the index data and the concentration data may be expressed on a nominal scale or an ordinal scale. In the case of the nominal or ordinal scale, any number may be allocated to each state for analysis.

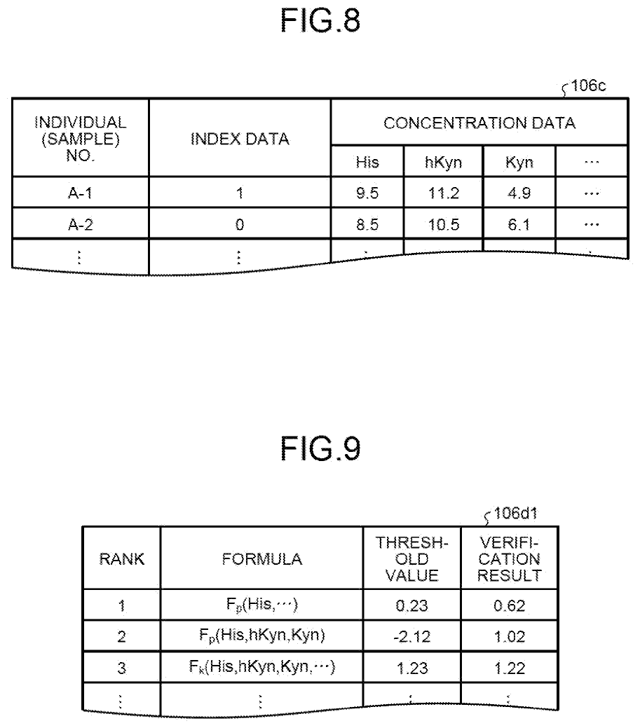

Returning to FIG. 5, the designated index state information file 106c stores the index state information designated in a designating part 102b described below. FIG. 8 is a chart showing an example of information stored in the designated index state information file 106c. As shown in FIG. 8, the information stored in the designated index state information file 106c includes the individual number, the designated index data, and the designated concentration data that are correlated to one another.

Returning to FIG. 5, the formula-related information database 106d is composed of the formula file 106dl storing the formula prepared in a formula-preparing part 102c described below. The formula file 106dl stores the formulae used for the evaluation. FIG. 9 is a chart showing an example of information stored in the formula file 106dl. As shown in FIG. 9, the information stored in the formula file 106dl includes a rank, the formula (e.g., Fₚ (His, . . . ), Fₚ (His, hKyn, Kyn), Fₖ (His, hKyn, Kyn, . . . ) in FIG. 9), a threshold corresponding to each formula-preparing method, and the verification result of each formula (e.g., the value of each formula) that are correlated to one another.

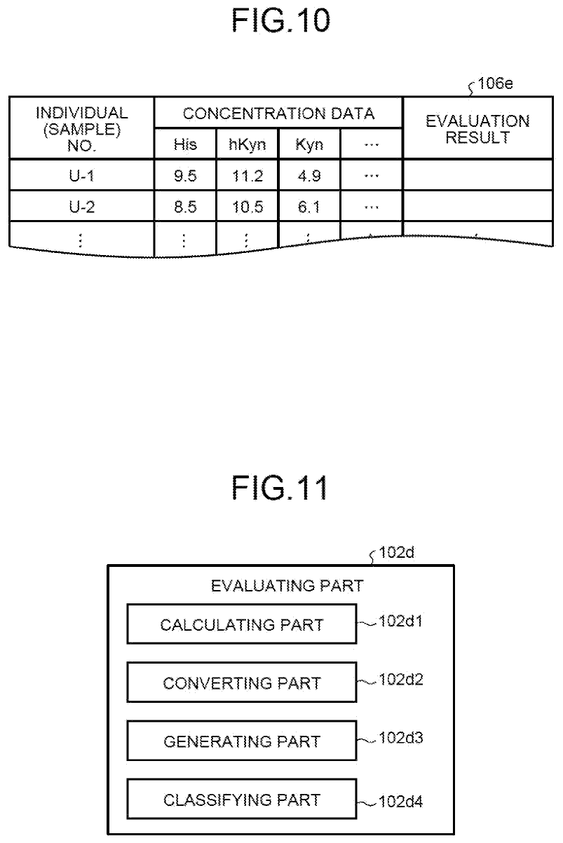

Returning to FIG. 5, the evaluation result file 106e stores the evaluation results obtained in the evaluating part 102d described later. FIG. 10 is a chart showing an example of the information stored in the evaluation result file 106e. The information stored in the evaluation result file 106e includes the individual number for uniquely identifying the individual (sample) as the subject to be evaluated, the previously obtained concentration data of the individual, and the evaluation result on the relative pharmacological action (the relative prognosis of treatment with combo therapy compared with the prognosis of treatment with monotherapy) that are correlated to one another. The evaluation result includes, for example, the value of the formula calculated by a calculating part 102dl described below, the converted value of the value of the formula obtained by a converting part 102d2 described below, the positional information generated by a generating part 102d3 described below, or the classification result obtained by a classifying part 102d4 described below.

Returning to FIG. 5, the control device 102 has an internal memory storing, for example, control programs such as OS (Operating System), programs for various processing procedures, and other needed data, and performs various information processings according to these programs. As shown in the figure, the control device 102 includes mainly an obtaining part 102a, the designating part 102b, the formula-preparing part 102c, the evaluating part 102d, a result outputting part 102e, and a sending part 102f. The control device 102 performs data processings such as removal of data including defective, removal of data including many outliers, and removal of explanatory variables for the defective-including data in the index state information transmitted from the database apparatus 400 and in the concentration data transmitted from the client apparatus 200.

The obtaining part 102a obtains information (specifically, concentration data, index state information, formula, and the like). For example, the obtaining part 102a may receive information (specifically, concentration data, index state information, formula, and the like) transmitted from the client apparatus 200 or the database apparatus 400 via the network 300 to obtain information. The obtaining part 102a may receive data for use in evaluation transmitted from a client apparatus 200 different from the client apparatus 200 to which the evaluation result is transmitted. When the evaluating apparatus 100 includes a mechanism (including hardware and software) for reading information recorded on a recording medium, the obtaining part 102a may obtain information by reading information recorded on a recording medium (specifically, concentration data, index state information, formula, and the like) through the mechanism. The specifying part 102b specifies target index data, concentration data, and combination presence/absence data in creating a formula.

The formula-preparing part 102c generates the formula based on the index state information obtained in the obtaining part 102a or the index state information designated in the designating part 102b. If the formulae are stored previously in a predetermined region of the memory device 106, the formula-preparing part 102c may generate the formula by selecting the desired formula out of the memory device 106. Alternatively, the formula-preparing part 102c may generate the formula by selecting and downloading the desired formula from another computer apparatus (e.g., the database apparatus 400) in which the formulae are previously stored.

The evaluating part 102d evaluates the relative pharmacological action in the individual by calculating a value of the formula in the presence of use of an anticancer drug as a combination drug and a value of the formula in the absence of the use, using the previously obtained formula (for example, the formula prepared by the formula-preparing part 102c or the formula obtained by the obtaining part 102a) and the concentration value included in the concentration data of the individual obtained by the obtaining part 102a. The evaluating part 102d may evaluate the relative pharmacological action in the individual using the concentration value included in the concentration data, the ratio or the difference of concentration values, or the converted value thereof (for example, the concentration standard score).

Hereinafter, a configuration of the evaluating part 102d will be described with reference to FIG. 11. FIG. 11 is a block diagram showing the configuration of the evaluating part 102d, and only a part in the configuration related to the present invention is shown conceptually. The evaluating part 102d includes the calculating part 102d1, the converting part 102d2, the generating part 102d3, and the classifying part 102d4, additionally.

The calculating part 102dl calculates a value of the formula in the presence of use of an anticancer drug as a combination drug and a value of the formula in the absence of the use, using the concentration value (which may be the ratio or the difference) included in the concentration data, and the formula at least including the explanatory variable to be substituted with the concentration value and the combination presence/absence variable. The evaluating part 102d may store the value of the formula calculated by the calculating part 102dl as the evaluation result in a predetermined region of the evaluation result file 106e.

The converting part 102d2 converts the value of the formula calculated by the calculating part 102d1, for example, by the conversion method described above. The evaluating part 102d may store the converted value by the converting part 102d2 as the evaluation result in a predetermined region of the evaluation result file 106e. The converting part 102d2 may convert the concentration value included in the concentration data, or the ratio or the difference of the concentration values, for example, by the conversion method described above.

The generating part 102d3 generates the positional information about the position of the predetermined mark on the predetermined scale visually presented on the display device such as a monitor or the physical medium such as paper, using the value of the formula calculated by the calculating part 102dl or the converted value by the converting part 102d2 (the concentration value, the ratio or the difference of the concentration values, or the converted value thereof may be used as well). The evaluating part 102d may store the positional information generated by the generating part 102d3 as the evaluation result in a predetermined region of the evaluation result file 106e.

The classifying part 102d4 classifies the individual into any one of the categories defined at least in consideration of the relative prognosis of treatment with combo therapy compared with the prognosis of treatment with monotherapy, using the value of the formula calculated by the calculating part 102dl or the converted value by the converting part 102d2 (the concentration value, the ratio or the difference of the concentration values, or the converted value thereof may be used as well).

The result outputting part 102e outputs, into the output device 114, for example, the processing results in each processing part in the control device 102 (including the evaluation results obtained by the evaluating part 102d).

The sending part 102f transmits the evaluation results to the client apparatus 200 that is a sender of the concentration data of the individual, and transmits the formulae prepared in the evaluating apparatus 100 and the evaluation results to the database apparatus 400. The sending part 102f may transmit the evaluation result to a client apparatus 200 different from the client apparatus 200 that from which data for use in evaluation is transmitted.

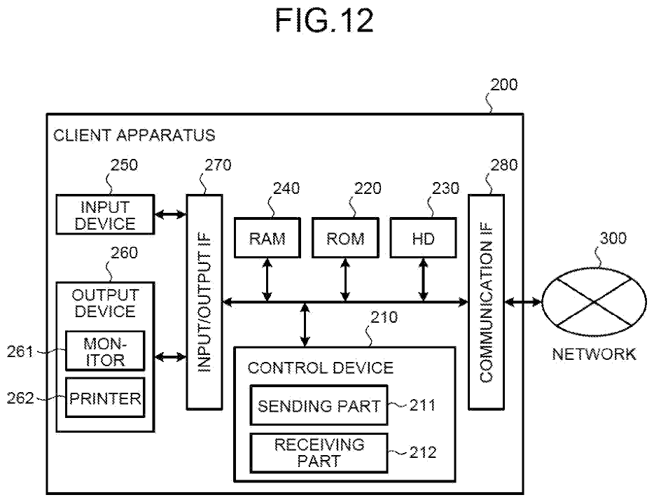

Hereinafter, a configuration of the client apparatus 200 in the present system will be described with reference to FIG. 12. FIG. 12 is a block diagram showing an example of the configuration of the client apparatus 200 in the present system, and only the part in the configuration relevant to the present invention is shown conceptually.

The client apparatus 200 includes a control device 210, ROM 220, HD (Hard Disk) 230, RAM 240, an input device 250, an output device 260, an input/output IF 270, and a communication IF 280 that are connected communicatively to one another through a communication channel. The client apparatus 200 may be realized based on an information processing apparatus (for example, an information processing terminal such as a known personal computer, a workstation, a family computer, Internet TV (Television), PHS (Personal Handyphone System) terminal, a mobile phone terminal, a mobile unit communication terminal, or PDA (Personal Digital Assistants)) connected as needed with peripheral devices such as a printer, a monitor, and an image scanner.

The input device 250 is, for example, a keyboard, a mouse, or a microphone. The monitor 261 described below also functions as a pointing device together with a mouse. The output device 260 is an output means for outputting information received via the communication IF 280, and includes the monitor 261 (including home television) and a printer 262. In addition, the output device 260 may have a speaker or the like additionally. The input/output IF 270 is connected to the input device 250 and the output device 260.

The communication IF 280 connects the client apparatus 200 to the network 300 (or communication apparatus such as a router) communicatively. In other words, the client apparatus 200 is connected to the network 300 via a communication apparatus such as a modem, TA (Terminal Adapter) or a router, and a telephone line, or via a private line. In this way, the client apparatus 200 can access to the evaluating apparatus 100 by using a particular protocol.

The control device 210 has a receiving part 211 and a sending part 212. The receiving part 211 receives various kinds of information such as the evaluation results transmitted from the evaluating apparatus 100, via the communication IF 280. The sending part 212 sends various kinds of information such as the concentration data of the individual, via the communication IF 280, to the evaluating apparatus 100.

All or a part of processings of the control device 210 may be performed by CPU and programs read and executed by the CPU. Computer programs for giving instructions to the CPU and executing various processings together with the OS (Operating System) are recorded in the ROM 220 or HD 230. The computer programs, which are executed as they are loaded in the RAM 240, constitute the control device 210 with the CPU. The computer programs may be stored in application program servers connected via any network to the client apparatus 200, and the client apparatus 200 may download all or a part of them as needed. All or any part of processings of the control device 210 may be realized by hardware such as wired-logic.

The control device 210 may include an evaluating part 210a (including a calculating part 210a1, a converting part 210a2, a generating part 210a3, and a classifying part 210a4) having the same functions as the functions of the evaluating part 102d in the evaluating apparatus 100. When the control device 210 includes the evaluating part 210a, the evaluating part 210a may convert the value of the formula (the concentration value, or the ratio or the difference of the concentration values may be used as well) in the converting part 210a2, generate the positional information corresponding to the value of the formula or the converted value (the concentration value, the ratio or the difference of the concentration values, or the converted value thereof may be used as well) in the generating part 210a3, and classify the individual into any one of the categories using the value of the formula or the converted value (the concentration value, the ratio or the difference of the concentration values, or the converted value thereof may be used as well) in the classifying part 210a4, in accordance with information included in the evaluation results transmitted from the evaluating apparatus 100.

Hereinafter, the network 300 in the present system will be described with reference to FIGS. 3 and 4. The network 300 has a function to connect the evaluating apparatus 100, the client apparatuses 200, and the database apparatus 400 mutually, communicatively to one another, and is for example the Internet, an intranet, or LAN (Local Area Network (including both wired and wireless)). The network 300 may be VAN (Value Added Network), a personal computer communication network, a public telephone network (including both analog and digital), a leased line network (including both analog and digital), CATV (Community Antenna Television) network, a portable switched network or a portable packet-switched network (including IMT2000 (International Mobile Telecommunication 2000) system, GSM (registered trademark) (Global System for Mobile Communications) system, or PDC (Personal Digital Cellular)/PDC-P system), a wireless calling network, a local wireless network such as Bluetooth (registered trademark), PHS network, a satellite communication network (including CS (Communication Satellite), BS (Broadcasting Satellite), ISDB (Integrated Services Digital Broadcasting) and the like), or the like.

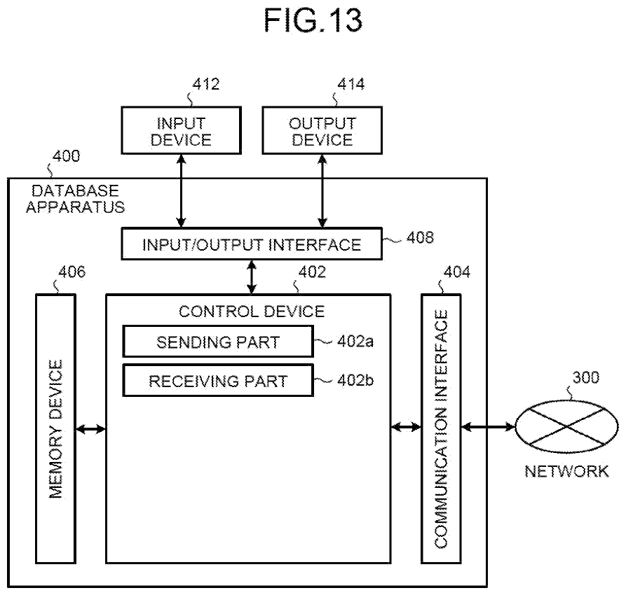

Hereinafter, the configuration of the database apparatus 400 in the present system will be described with reference to FIG. 13. FIG. 13 is a block diagram showing an example of the configuration of the database apparatus 400 in the present system, showing conceptually only the region relevant to the present invention.

The database apparatus 400 has functions to store, for example, the index state information used in preparing the formulae in the evaluating apparatus 100 or the database apparatus, the formulae prepared in the evaluating apparatus 100, and the evaluation results obtained in the evaluating apparatus 100. As shown in FIG. 13, the database apparatus 400 includes; (i) a control device 402, such as CPU, that integrally controls the database apparatus; (ii) a communication interface 404 connecting the database apparatus to the network 300 communicatively via communication apparatuses such as a router and wired or wireless communication circuits such as a private line; (iii) a memory device 406 storing various databases, tables, files (for example, files for Web pages) and others; and (iv) an input/output interface 408 connected to an input device 412 and an output device 414, and these parts are connected communicatively to each other via any communication channel.

The memory device 406 is a storage means, and, examples thereof include a memory apparatus such as RAM or ROM, a fixed disk drive such as a hard disk, a flexible disk, and an optical disk. The memory device 406 stores, for example, various programs used in various processings. The communication interface 404 allows communication between the database apparatus 400 and the network 300 (or a communication apparatus such as a router). Thus, the communication interface 404 has a function to communicate data via a communication line with other terminals. The input/output interface 408 is connected to the input device 412 and the output device 414. A monitor (including a home television), a speaker, or a printer may be used as the output device 414. A keyboard, a mouse, a microphone, or a monitor functioning as a pointing device together with a mouse may be used as the input device 412.

The control device 402 has an internal memory storing, for example, control programs such as OS (Operating System), programs for various processing procedures, and other needed data, and performs various information processings according to these programs. As shown in the figure, the control device 402 includes mainly a sending part 402a and a receiving part 402b. The sending part 402a transmits various kinds of information such as the index state information and the formulae to the evaluating apparatus 100. The receiving part 402b receives various kinds of information such as the formula and the evaluation results, transmitted from the evaluating apparatus 100.

In the present description, the evaluating apparatus 100 executes the obtainment of the concentration data, the calculation of the value of the formula, the classification of the individual into the category, and the transmission of the evaluation results, while the client apparatus 200 executes the reception of the evaluation results, described as an example. However, when the client apparatus 200 includes the evaluating unit 210a, the evaluating apparatus 100 only has to execute the calculation of the value of the formula. For example, the conversion of the value of the formula, the generation of the positional information, and the classification of the individual into the category may be appropriately shared between the evaluating apparatus 100 and the client apparatus 200.

For example, when the client apparatus 200 receives the value of the formula from the evaluating apparatus 100, the evaluating unit 210a may convert the value of the formula in the converting unit 210a2, generate the positional information corresponding to the value of the formula or the converted value in the generating unit 210a3, and classify the individual into any one of the categories using the value of the formula or the converted value in the classifying unit 210a4.

When the client apparatus 200 receives the converted value from the evaluating apparatus 100, the evaluating unit 210a may generate the positional information corresponding to the converted value in the generating unit 210a3, and classify the individual into any one of the categories using the converted value in the classifying unit 210a4.

When the client apparatus 200 receives the value of the formula or the converted value and the positional information from the evaluating apparatus 100, the evaluating unit 210a may classify the individual into any one of the categories using the value of the formula or the converted value in the classifying unit 210a4.

### 2-3. Other Embodiments

In addition to the second embodiment described above, the evaluating apparatus, the calculating apparatus, the evaluating method, the calculating method, the evaluating program, the calculating program, the recording medium, the evaluating system, and the terminal apparatus according to the present invention can be practiced in various different embodiments within the technological scope of the claims.

Of the processings described in the second embodiment, all or a part of the processings described as automatically performed ones may be manually performed, or all or a part of the processings described as manually performed ones may be also automatically performed by known methods.

In addition, the processing procedures, the control procedures, the specific names, the information including parameters such as registered data of various processings and retrieval conditions, the screen examples, and the database configuration shown in the description and the drawings may be arbitrarily modified unless otherwise specified.

The components of the evaluating apparatus 100 shown in the figures are functionally conceptual and therefore not be physically configured as shown in the figures.

For example, for the operational functions provided in the evaluating apparatus 100, in particular, for the operational functions performed in the control device 102, all or part thereof may be implemented by the CPU (Central Processing Unit) and programs interpreted and executed in the CPU, or may be implemented by wired-logic hardware. The program is recorded in a non-transitory tangible computer-readable recording medium including programmed instructions for making an information processing apparatus execute the evaluating method or the calculating method according to the present invention, and is mechanically read as needed by the evaluating apparatus 100. More specifically, computer programs to give instructions to the CPU in cooperation with the OS (operating system) to perform various processes are recorded in the memory device 106 such as ROM or a HDD (hard disk drive). The computer programs are executed by being loaded to RAM, and form the control unit in cooperation with the CPU.

The computer programs may be stored in an application program server connected to the evaluating apparatus 100 via an arbitrary network, and all or part thereof can be downloaded as necessary.

The evaluating program or the calculating program according to the present invention may be stored in the non-transitory tangible computer-readable recording medium, or can be configured as a program product. The “recording medium” mentioned here includes any “portable physical medium” such as a memory card, a USB (universal serial bus) memory, an SD (secure digital) card, a flexible disk, a magneto-optical disc, ROM, EPROM (erasable programmable read only memory), EEPROM (registered trademark) (electronically erasable and programmable read only memory) CD-ROM (compact disk read only memory), MO (magneto-optical disk), DVD (digital versatile disk), and Blu-ray (registered trademark) Disc.

The “program” mentioned here is a data processing method described in an arbitrary language or description method, and therefore any form such as a source code and a binary code is acceptable. The “program” is not necessarily limited to a program configured as a single unit, and, therefore, includes those dispersively configured as a plurality of modules and libraries and those in which the function of the program is achieved in cooperation with separate programs represented as OS (operating system). Any known configuration and procedures can be used as a specific configuration and reading procedure to read a recording medium by each apparatus shown in the embodiments, an installation procedure after the reading, and the like.

The various databases and the like stored in the memory device 106 is a storage unit such as a memory device such as RAM and ROM, a fixed disk drive such as a hard disk, a flexible disk, or an optical disc. The memory device 106 stores therein various programs, tables, databases, files for Web (World Wide Web) pages, and the like used to perform various processes and to provide Web sites.

The evaluating apparatus 100 may be configured as an information processing apparatus such as known personal computer and work station, or may be configured as the information processing apparatus connected to an arbitrary peripheral device. The evaluating apparatus 100 may be provided by installing software (including the programs and the data, etc.) to cause the information processing apparatus to implement the evaluating method or the calculating method according to the present invention.

Furthermore, a specific configuration of dispersion or integration of the apparatuses is not limited to the shown one. The apparatuses can be configured by functionally or physically dispersing or integrating all or part of the apparatuses in arbitrary units according to various types of additions or the like or according to functional loads. In other words, the embodiments may be implemented in arbitrary combinations thereof or an embodiment may be selectively implemented.

### Example 1

A study was conducted to optimize the ICI treatment patient stratification technique using amino acid profiles (jRCT1031190196). Specifically, multivariate discriminants that can be used to predict the prognosis before treatment or at an early stage after the start of treatment or to choose treatment were created by selecting the most appropriate combination of parameters by analyzing the correlation of the measured concentration values of amino acids and amino acid-related metabolites with clinical data (patient background, tumor shrinkage (RECIST Ver. 1.1), progression-free survival (PFS), overall survival (OS), and side effects (adverse events)), molecular pathological findings, and the like.

For 104 patients with advanced or recurrent non-small cell lung cancer who were treated with ICI monotherapy or chemotherapy combo therapy with an ICI and an anticancer drug as a combination drug, 5 mL of blood samples were taken before the start of treatment and 6 weeks after the start of treatment. Patient background information, disease background, tumor information, treatment information, physical examination information, blood test information, and treatment prognosis information (tumor shrinkage, progression-free survival, overall survival, and side effects) were also obtained as medical information from all the target patients. All the target patients had not consumed amino acid supplements or sports drinks containing amino acids and had not exercised excessively since the day before blood samples were taken. All the target patients had fasted for at least 10 hours after dinner the day before blood samples were taken. Blood samples were taken in the morning on an empty stomach using a vacuum blood collection tube (5-mL blood collection tube with EDTA-2Na).

The concentration values of the following 21 kinds of amino acids and the following 8 kinds of amino acid-related metabolites were measured using the collected blood samples. Specifically, plasma was separated immediately from the collected blood samples, and the resulting plasma samples were stored in an ultra-low temperature freezer.

At the time of measuring the concentration values, the plasma samples were subjected to a series of processing including thawing, deproteinization, and dilution, and the concentration values of amino acids and amino acid-related metabolites were measured using an LC-MS analyzer or an LC-MS/MS analyzer.

### [Amino Acids: 21 Kinds]

Glutamic Acid, Arginine, Ornithine, Citrulline, Histidine, Valine, Phenylalanine, Tyrosine, Methionine, Proline, Asparagine, Leucine, Lysine, Threonine, Isoleucine, Glutamine, Alanine, Serine, α-Aminobutyric acid, Tryptophan, and Glycine

### [Amino Acid-Related Metabolites: 8 Kinds]

Anthranilic Acid, 3-Hydroxyl Kynurenine, 5-Hydroxyl-Tryptophan, Kynurenine, Kynurenic Acid, Neopterin, Quinolinic Acid, and Xanthurenic Acid

Of all the target patients for whom concentration values were measured, 96 patients who met the eligibility criteria and conformed to the data acquisition procedure underwent analysis using the concentration values and the medical information. Specifically, multivariate discriminants for predicting OS after ICI treatment were created using the following procedure A) to E). The overall population (96 patients) included a subgroup treated with ICI monotherapy (32 cases) and a subgroup treated with chemotherapy combo therapy (64 cases). The kinds of drugs for each subgroup were as follows.

### [ICI Monotherapy: 32 Cases]

Atezolizumab: 3 cases
Pembrolizumab: 19 cases
Nivolumab: 9 cases
Nivolumab+ipilimumab: 1 case

### [Chemotherapy Combo Therapy: 64 Cases]

Atezolizumab, carboplatin, and nab-paclitaxel: 2 cases
Atezolizumab, carboplatin, paclitaxel, and bevacizumab: 10 cases
Atezolizumab, carboplatin, and pemetrexed: 3 cases
Atezolizumab, carboplatin, pemetrexed, and bevacizumab: 2 cases
Pembrolizumab, carboplatin, and nab-paclitaxel: 9 cases
Pembrolizumab, carboplatin, and paclitaxel: 7 cases
Pembrolizumab, carboplatin, and pemetrexed: 19 cases
Pembrolizumab, cisplatin, pemetrexed: 9 cases
Nivolumab, carboplatin, pemetrexed, and ipilimumab: 3 cases

### A) Analysis of Univariate Correlation

Correlation analysis between OS and the plasma concentrations of amino acids and amino acid-related metabolites is performed based on OS survival time analysis using the Cox hazard model for the whole population and each of the two subgroups. The p-value, the hazard ratio, and ROC_AUC are calculated as the results of the correlation analysis.

### (B) Selection of Parameters

The plasma concentrations of amino acids or amino acid-related metabolites are selected as candidates for parameters to be set in multivariate discriminants, using the results of the correlation analysis and information as covariates, such as the presence or absence of the combination with an anticancer drug.

### (C) Creation of Multivariate Discriminants

Analysis based on the Cox hazard model is performed using the selected plasma concentrations, the presence or absence of the combination with an anticancer drug, and the product term of the plasma concentration and the presence or absence of the combination. The p-value, AIC/BIC, C-index, and the hazard ratio and the 95% confidence interval thereof are calculated as the results of the analysis. The lower limit of the 95% confidence interval of ROC_AUC serving as a criterion for OS discriminating performance at each time point or the lower limit of the 95% confidence interval of C-index serving as a criterion for OS predicting performance is calculated using leave-one-out cross-validation, split cross-validation, or bootstrapping. Candidate multivariate discriminants are selected using the calculated values, based on the statistical significance of the model.

### (D) Confirmation of Discriminating Performance

In the whole population and each of the two subgroups for which multivariate discriminants are created, cutoff values for multivariate discriminants are determined by referring to Youden index criteria, (0,1)-closest method, DP plot analysis, or the like. For comparison on the OS survival time prediction between the discrimination groups, the hazard ratio and the 95% confidence interval thereof, the p-value of the Cox hazard test, the p-value of the log-rank test, the hazard ratio, and the median survival time and the confidence interval thereof for each group are calculated.

### (E) Application of Multivariate Discriminants (Subgroup Analysis)

The multivariate discriminants and the cutoff values created for the whole population and each of the two subgroups are mutually applied to test the statistics for intergroup comparison in each population. For each group determined (positive and negative), the comparison of OS depending on the presence or absence of the combination with an anticancer drug, the estimation of interactions, and the testing of statistical significance are performed. Stratified analysis by patient background (age, gender, histological type, progression, PS and PD-L1 expression, etc.), ICI/anticancer drug treatment regimen (with or without ipilimumab, etc.), and enrolling medical institution, as well as multivariate analysis for OS discrimination is performed. The discriminating performance of the multivariate discriminants for other endpoints, namely, tumor shrinkage and PFS, is also confirmed.

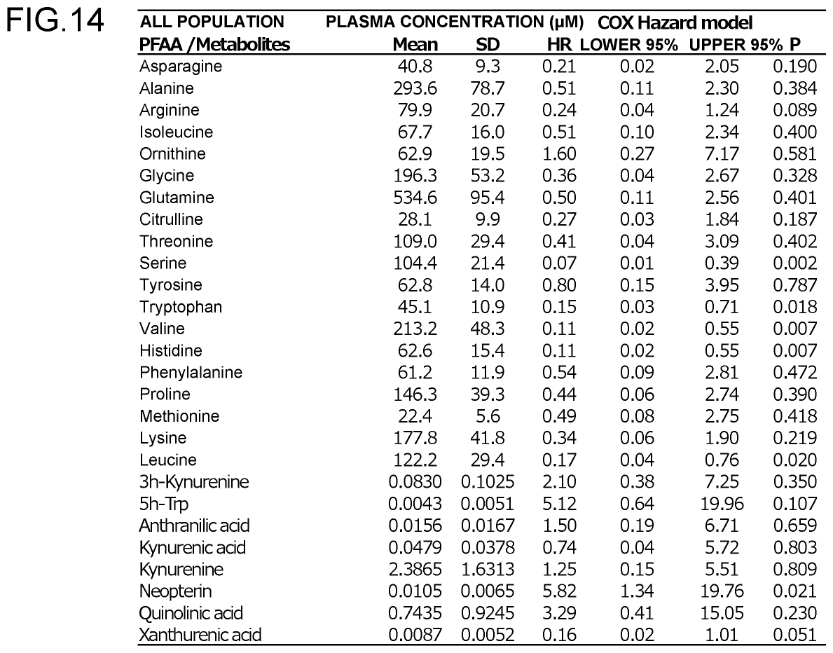

The results of the analysis will be explained below. FIG. 14 is a table showing the results of the correlation analysis between the measurement values of amino acids and amino acid-related metabolites in plasma before the start of treatment and the prognosis of treatment (OS) for the whole population. Eight kinds, namely, asparagine, arginine, citrulline, serine, tryptophan, valine, histidine, and leucine were identified as amino acids with significant change, and three kinds, namely, 5h-Trp, Neopterin, and Xanthurenic Acid were identified as amino acid-related metabolites with significant change. It was shown that the blood concentration values of the eight kinds of amino acids and the three kinds of amino acid-related metabolites before the start of treatment could be indicators for predicting the prognosis of treatment with an ICI (specifically, the prognosis of treatment regardless of the presence or absence of the combination with an anticancer drug (for example, whether the prognosis of treatment is good or poor with either monotherapy or combo therapy)).

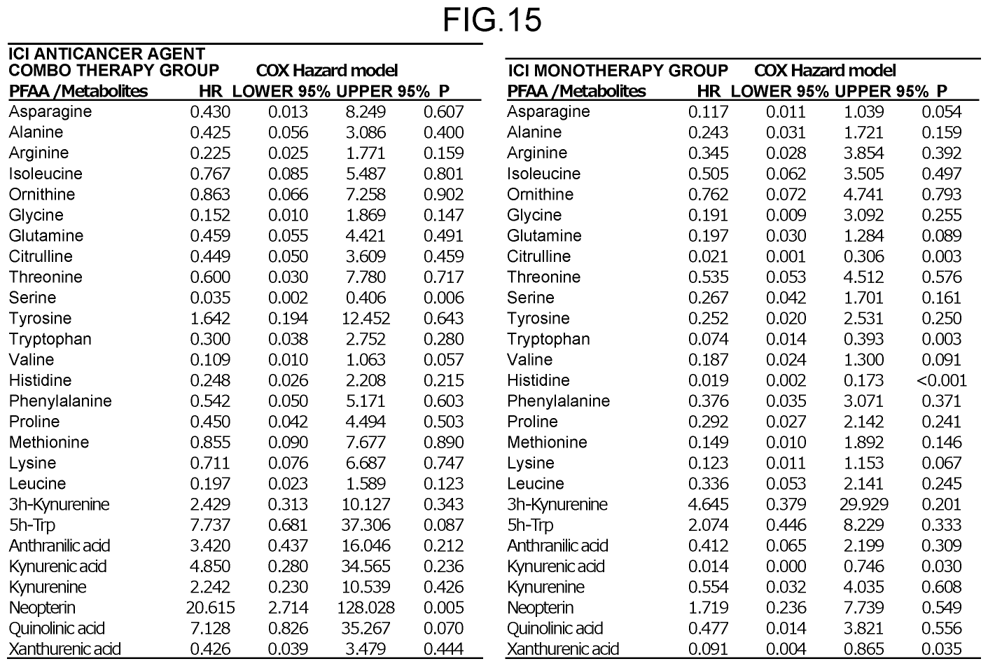

FIG. 15 is a table showing the results of the correlation analysis between the measurement values of amino acids and amino acid-related metabolites in plasma before the start of treatment and the prognosis of treatment (OS) for the monotherapy subgroup. Ten kinds, namely, asparagine, alanine, glutamine, citrulline, serine, tryptophan, valine, histidine, methionine, and lysine were identified as amino acids with significant change, and two kinds, namely, Kynurenic Acid and Xanthurenic acid were identified as amino acid-related metabolites with significant change. It was shown that the blood concentration values of the ten kinds of amino acids and the two kinds of amino acid-related metabolites before the start of treatment could be indicators for predicting the prognosis of treatment with monotherapy.

FIG. 15 is a table showing the results of the correlation analysis between the measurement values of amino acids and amino acid-related metabolites in plasma before the start of treatment and the prognosis of treatment (OS) for the combo therapy subgroup. Five kinds, namely, arginine, glycine, serine, valine, and leucine were identified as amino acids with significant change, and three kinds, namely, 5h-Trp, Neopterin, and Quinolinic acid were identified as amino acid-related metabolites with significant change. It was shown that the blood concentration values of the five kinds of amino acids and the three kinds of amino acid-related metabolites before the start of treatment could be indicators for predicting the prognosis of treatment with combo therapy.

Multivariate discriminants based on a covariate model and multivariate discriminants based on a stratified model were created to be used as indicators for comparing the prognosis of treatment with monotherapy and the prognosis of treatment with combo therapy. Specifically, the covariant, that is, the presence or absence of the combination was incorporated as a dummy explanatory variable into the multivariate discriminant and optimized to the following multivariate discriminant (formula F) for predicting (discriminating) the prognosis of treatment (OS) for the whole population.

F=intercept+coefficient a×amino acid A+coefficient b×amino acid B+coefficient c×presence or absence of combination+coefficient d×presence or absence of combination×amino acid B
When formula F is a covariate model, the part “coefficient c×presence or absence of combination+coefficient d×presence or absence of combination×amino acid B” is the part that is adjusted by the dummy explanatory variable on the presence or absence of the combination. When formula F is a stratified model, formula F does not include the term “coefficient c×presence or absence of combination”, and the terms “intercept” and “coefficient d×presence or absence of combination×amino acid B” are the parts that are adjusted by the presence or absence of the combination.
In formula F, “amino acid A” and “amino acid B” are explanatory variables to be substituted with the concentration values of amino acids and amino acid-related metabolites, respectively.
In formula F, “presence or absence of combination” is a dummy explanatory variable that takes a value of 0 in the absence of the combination and takes a value of 1 in the presence of the combination.

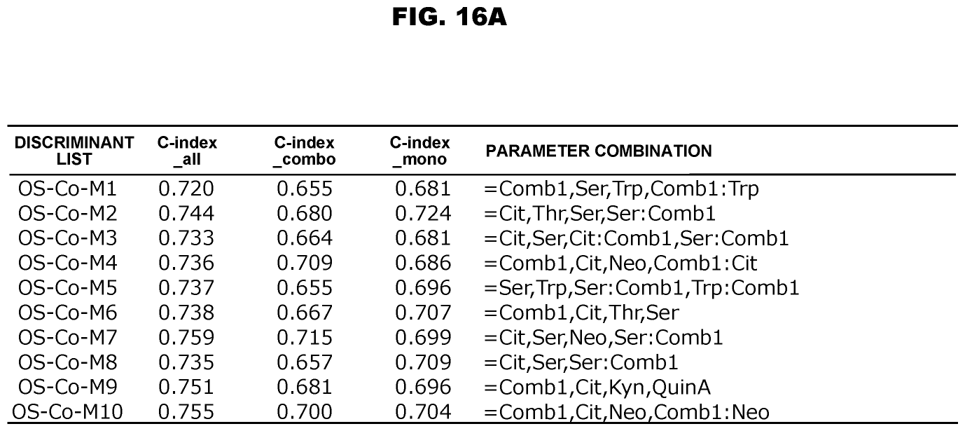

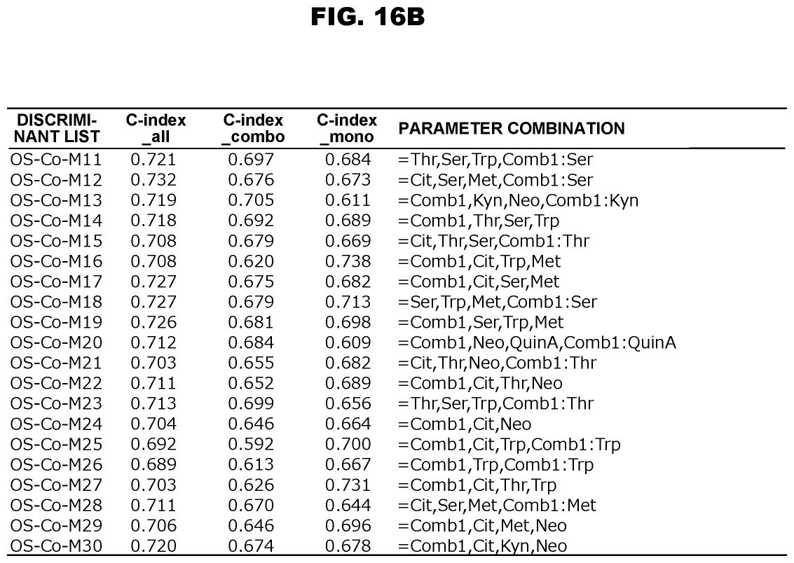

Information on multivariate discriminants based on the covariate model is shown in FIG. 16A and FIG. 16B. FIG. 16A is a table showing the multivariate discriminants created using data sets at a minimum of six months of follow-up (median patient follow-up time of 250 days) for all the participants in study, including the last target patient enrolled in the study. FIG. 16B is a table showing the multivariate discriminants created using data sets at a minimum of one year of follow-up (median patient follow-up time of 359 days) for all the participants in study, including the last target patient enrolled in the study. Since the survival time varies with therapeutic effects, the data sets for discriminants were prepared by delimiting the overall follow-up when the last patient enrolled in the study had met the minimum follow-up time. In FIG. 16A and FIG. 16B, the column “C-index_all” shows the C-index for the whole population, the column “C-index_combo” shows the C-index for the combo therapy subgroup, and the column “C-index_mono” shows the C-index for the monotherapy subgroup. In FIG. 16A and FIG. 16B, “Comb1” included in the multivariate discriminants represents the dummy explanatory variable on the presence or absence of the combination. In FIG. 16A and FIG. 16B, the symbol “:” represents the multiplication sign.

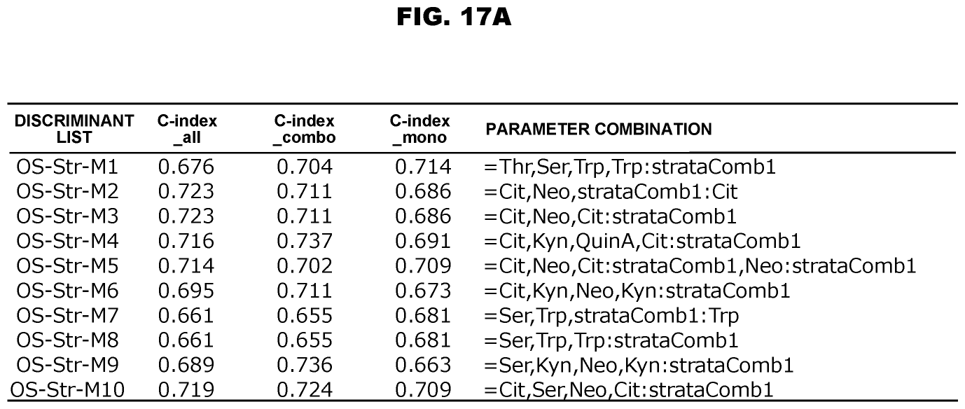

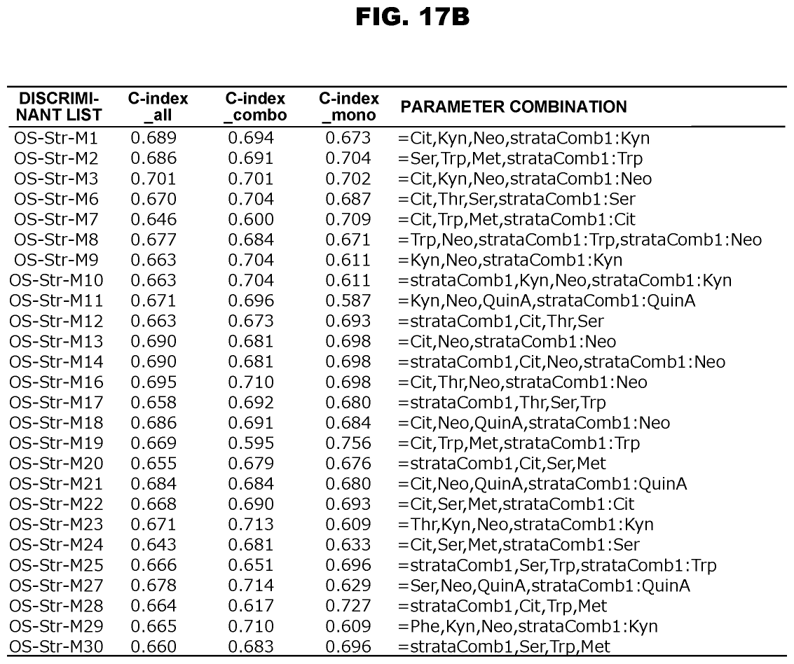

Information on multivariate discriminants based on a stratified model is shown in FIG. 17A and FIG. 17B. FIG. 17A is a table showing the multivariate discriminants created using data sets at a minimum of six months of follow-up (median patient follow-up time of 250 days) for all the participants in study, including the last target patient enrolled in the study. FIG. 17B is a table showing the multivariate discriminants created using data sets at a minimum of one year of follow-up (median patient follow-up time of 359 days) for all the participants in study, including the last target patient enrolled in the study. Since the survival time varies with therapeutic effects, the data sets for discriminants were prepared by delimiting the overall follow-up when the last patient enrolled in the study had met the minimum follow-up time. In FIG. 17A and FIG. 17B, the column “C-index_all” shows the C-index for the whole population, the column “C-index_combo” shows the C-index for the combo therapy subgroup, and the column “C-index_mono” shows the C-index for the monotherapy subgroup. In FIG. 17A and FIG. 17B, “strataComb1” included in the multivariate discriminants represents the dummy explanatory variable on the presence or absence of the combination. In FIG. 17A and FIG. 17B, the symbol “:” represents the multiplication sign.

The multivariate discriminants listed in FIG. 16A, FIG. 16B, FIG. 17A, and FIG. 17B were used to calculate a risk score in the absence of the combination (hereinafter referred to as the “monotherapy risk score”) and a risk score in the presence of the combination (hereinafter referred to as “combo therapy risk score”) for one patient. The prognosis of treatment with monotherapy and the prognosis of treatment with combo therapy were compared and evaluated according to “monotherapy risk score—combo therapy risk score” (hereinafter referred to as “differential risk score”).

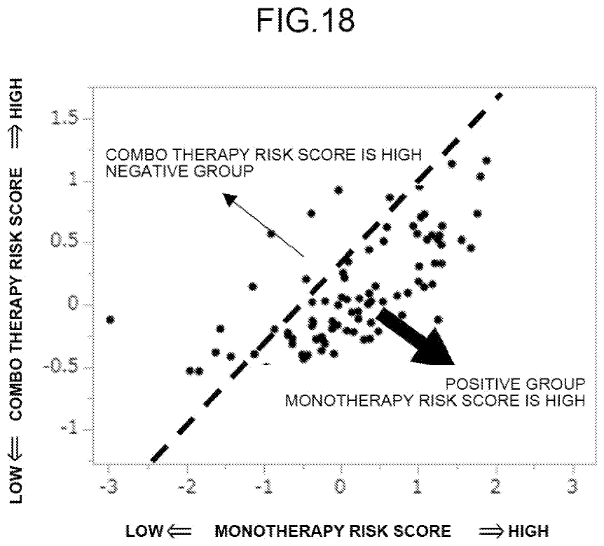

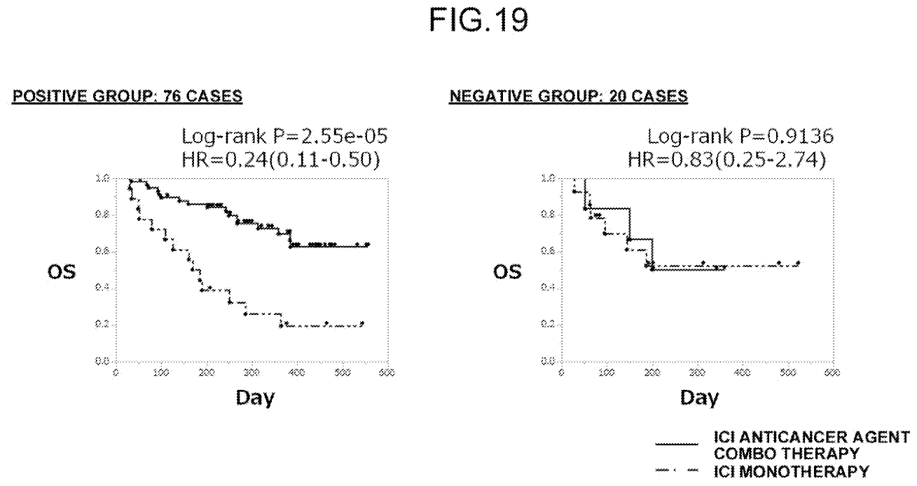

FIG. 18 is a chart showing a distribution of monotherapy risk score and combo therapy risk score for each patient calculated using the multivariate discriminant “OS-Co-M3” listed in FIG. 16A for the whole population. Classification was performed into a positive group positioned at the lower right side of the diagonal line where the differential risk score was greater than the cutoff value, and a negative group positioned at the upper left side of the diagonal line where the differential risk score was smaller than the cutoff value. FIG. 19 is a chart showing survival time curves by treatment for the positive group and survival time curves by treatment for the negative group. In FIG. 19, “Mono” represents actual monotherapy prognostic data and “Combo” represents combo therapy prognostic data. In the positive group, the prognosis of treatment with monotherapy is poor, and the combo therapy is expected to be more therapeutically effective than the monotherapy. In other words, it was shown that the therapeutic effect (additional effect) of combo therapy compared with the therapeutic effect of monotherapy can be predicted by the difference between the two kinds of scores obtained from this multivariate discriminant.

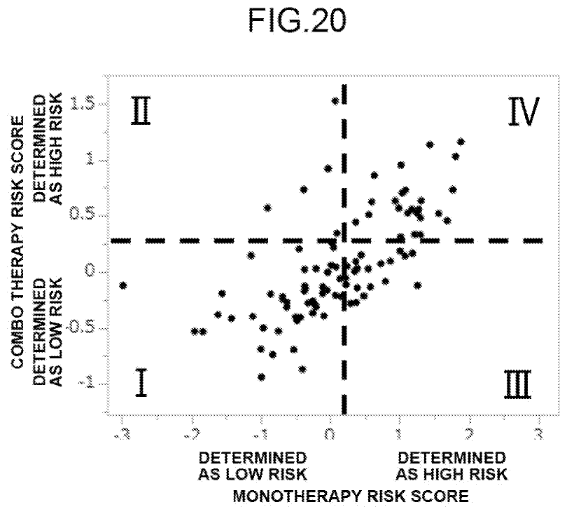

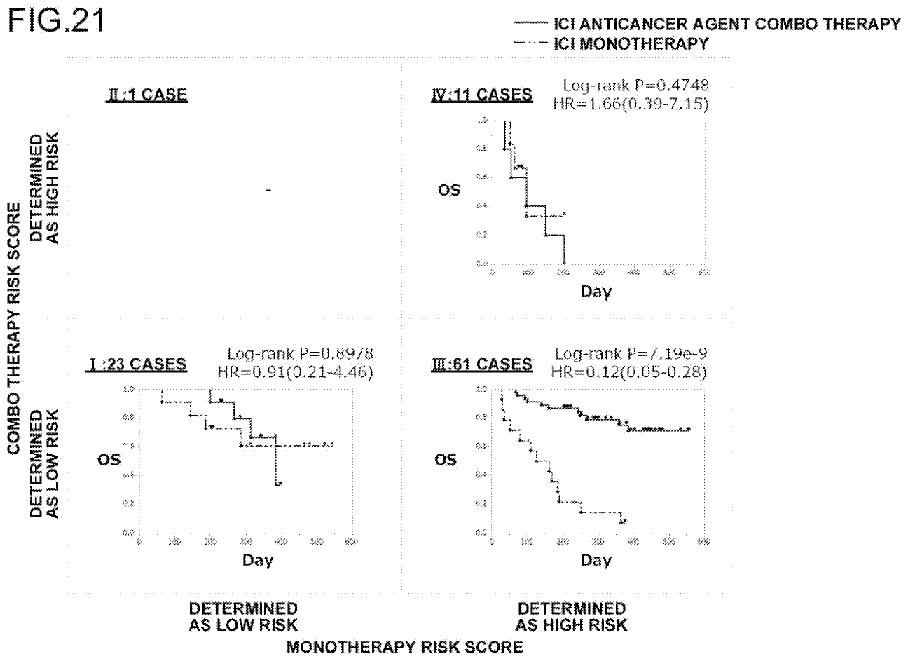

FIG. 20 is a chart showing a distribution of monotherapy risk score and combo therapy risk score for each patient calculated using the multivariate discriminant “OS-Co-M3” listed in FIG. 16A for the whole population. The cutoff value for the monotherapy risk score and the cutoff value for the combo therapy risk score were set, and a risk score equal to or greater than the cutoff value was determined as high risk and a risk score smaller than the cutoff value was determined as low risk. The whole population was classified into Group I in which both the monotherapy risk score and the combo therapy risk score were determined as low risk (good prognosis), Group II in which the monotherapy risk score was determined as low risk (good prognosis) and the combo therapy risk score was determined as high risk (poor prognosis), Group III in which the monotherapy risk score was determined as high risk (poor prognosis) and the combo therapy risk score was determined as low risk (good prognosis), and Group IV in which both the monotherapy risk score and the combo therapy risk score were determined as high risk (poor prognosis). FIG. 21 is a chart showing survival time curves by treatment for each group. In FIG. 21, “Mono” represents monotherapy and “Combo” represents combo therapy. In Group I, both therapies were confirmed to have a good prognosis. Group II is a group in which the prognosis of monotherapy is expected to exceed that of combo therapy, but patients in this group were considered to be rare. In Group III, the prognosis of treatment with monotherapy is poor, and the combo therapy is expected to be more therapeutically effective than the monotherapy. In Group IV, both therapies had a poor prognosis, suggesting that other treatment options should be considered. In other words, it was shown that the therapeutic effect (additional effect) of combo therapy compared with the therapeutic effect of monotherapy can be predicted by the combination of the two kinds of scores obtained from this multivariate discriminant.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An evaluating method comprising:
an evaluating step of evaluating a relative pharmacological action of a combination of an immune checkpoint inhibitor and an anticancer drug as a combination drug compared with a pharmacological action of an immune checkpoint inhibitor alone in a subject to be evaluated, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hKyn, hTrp, Kyn, KynA, NP, QA, and XA in blood of the subject to be evaluated, or (ii) a value of a formula in the absence of use of an anticancer drug as a combination drug and a value of the formula in the presence of the use that are calculated using the concentration value and the formula including an explanatory variable to be substituted with the concentration value and an explanatory variable on the presence or absence of the use.

2. The evaluating method according to claim 1, wherein in the evaluating step, the relative pharmacological action in the subject to be evaluated is evaluated using a difference between the value of the formula in the absence of the use and the value of the formula in the presence of the use.

3. The evaluating method according to claim 1, wherein in the evaluating step, the relative pharmacological action in the subject to be evaluated is evaluated using a combination of a result of evaluating a pharmacological action of an immune checkpoint inhibitor alone in the subject to be evaluated using the value of the formula in the absence of the use and a result of evaluating a pharmacological action of the combination in the subject to be evaluated using the value of the formula in the presence of the use.

4. The evaluating method according to claim 1, wherein the blood is taken from the subject to be evaluated after or before treatment with an immune checkpoint inhibitor or treatment with an anticancer drug used as a combination drug with an immune checkpoint inhibitor is started, and in the evaluating step, a relative effect of treatment with the combination compared with an effect of treatment with an immune checkpoint inhibitor alone in the subject to be evaluated is evaluated.

5. The evaluating method according to claim 1, wherein the evaluating step is performed by a control unit of an information processing apparatus including the control unit.

6. A calculating method comprising:
a calculating step of calculating a value of a formula for evaluating a relative pharmacological action of a combination of an immune checkpoint inhibitor and an anticancer drug as a combination drug compared with a pharmacological action of an immune checkpoint inhibitor alone in the absence of use of an anticancer drug as a combination drug and a value of the formula in the presence of the use, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hKyn, hTrp, Kyn, KynA, NP, QA, and XA in blood of a subject to be evaluated, and (ii) the formula including an explanatory variable to be substituted with the concentration value and an explanatory variable on the presence or absence of the use.

7. The calculating method according to claim 6, wherein the blood is taken from the subject to be evaluated after or before treatment with an immune checkpoint inhibitor or treatment with an anticancer drug used as a combination drug with an immune checkpoint inhibitor is started, and the formula is to evaluate a relative effect of treatment with the combination compared with an effect of treatment with an immune checkpoint inhibitor alone.

8. The calculating method according to claim 6, wherein the calculating step is performed by a control unit of an information processing apparatus including the control unit.

9. An evaluating apparatus comprising a control unit, wherein the control unit includes:
an evaluating unit that evaluates a relative pharmacological action of a combination of an immune checkpoint inhibitor and an anticancer drug as a combination drug compared with a pharmacological action of an immune checkpoint inhibitor alone in a subject to be evaluated, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hKyn, hTrp, Kyn, KynA, NP, QA, and XA in blood of the subject to be evaluated, or (ii) a value of a formula in the absence of use of an anticancer drug as a combination drug and a value of the formula in the presence of the use that are calculated using the concentration value and the formula including an explanatory variable to be substituted with the concentration value and an explanatory variable on the presence or absence of the use.

10. The evaluating apparatus according to claim 9, being communicatively connected via a network to a terminal apparatus that provides concentration data on the concentration value or the value of the formula,
wherein the control unit further includes:
a data receiving unit that receives the concentration data or the value of the formula transmitted from the terminal apparatus; and
a result transmitting unit that transmits an evaluation result obtained by the evaluating unit to the terminal apparatus,
wherein the evaluating unit uses the concentration value included in the concentration data or the value of the formula received by the data receiving unit.

11. A calculating apparatus comprising a control unit, wherein the control unit includes:
a calculating unit that calculates a value of a formula for evaluating a relative pharmacological action of a combination of an immune checkpoint inhibitor and an anticancer drug as a combination drug compared with a pharmacological action of an immune checkpoint inhibitor alone in the absence of use of an anticancer drug as a combination drug and a value of the formula in the presence of the use, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hKyn, hTrp, Kyn, KynA, NP, QA, and XA in blood of a subject to be evaluated, and (ii) the formula including an explanatory variable to be substituted with the concentration value and an explanatory variable on the presence or absence of the use.

12. An evaluating program for causing an information processing apparatus including a control unit to perform an evaluating method,
wherein the evaluating method includes:
an evaluating step of evaluating a relative pharmacological action of a combination of an immune checkpoint inhibitor and an anticancer drug as a combination drug compared with a pharmacological action of an immune checkpoint inhibitor alone in a subject to be evaluated, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hKyn, hTrp, Kyn, KynA, NP, QA, and XA in blood of the subject to be evaluated, or (ii) a value of a formula in the absence of use of an anticancer drug as a combination drug and a value of the formula in the presence of the use that are calculated using the concentration value and the formula including an explanatory variable to be substituted with the concentration value and an explanatory variable on the presence or absence of the use.

13. A calculating program for causing an information processing apparatus including a control unit to perform a calculating method,
wherein the calculating method includes:
a calculating step of calculating a value of a formula for evaluating a relative pharmacological action of a combination of an immune checkpoint inhibitor and an anticancer drug as a combination drug compared with a pharmacological action of an immune checkpoint inhibitor alone in the absence of use of an anticancer drug as a combination drug and a value of the formula in the presence of the use, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hKyn, hTrp, Kyn, KynA, NP, QA, and XA in blood of a subject to be evaluated, and (ii) the formula including an explanatory variable to be substituted with the concentration value and an explanatory variable on the presence or absence of the use.

14. A computer readable recording medium storing the program according to claim 12 or 13.

15. An evaluating system comprising:
an evaluating apparatus including a control unit; and
a terminal apparatus including a control unit,
wherein the evaluating apparatus and the terminal apparatus are communicatively connected to each other via a network,
the control unit of the terminal apparatus includes:
(I) a data transmitting unit that transmits, to the evaluating apparatus, (i) concentration data on a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hKyn, hTrp, Kyn, KynA, NP, QA, and XA in blood of a subject to be evaluated, or (ii) a value of a formula in the absence of use of an anticancer drug as a combination drug and a value of the formula in the presence of the use that are calculated using the concentration value and the formula including an explanatory variable to be substituted with the concentration value and an explanatory variable on the presence or absence of the use; and
(II) a result receiving unit that receives an evaluation result transmitted from the evaluating apparatus on a relative pharmacological action of a combination of an immune checkpoint inhibitor and an anticancer drug as a combination drug compared with a pharmacological action of an immune checkpoint inhibitor alone, and
the control unit of the evaluating apparatus includes:
(I) a data receiving unit that receives the concentration data or the value of the formula transmitted from the terminal apparatus;
(II) an evaluating unit that evaluates the relative pharmacological action of the combination compared with a pharmacological action of an immune checkpoint inhibitor alone in the subject to be evaluated, using the concentration value included in the concentration data or the value of the formula received by the data receiving unit; and
(III) a result transmitting unit that transmits the evaluation result obtained by the evaluating unit to the terminal apparatus.

16. A terminal apparatus comprising a control unit, wherein the control unit includes:
a result obtaining unit that obtains an evaluation result on a relative pharmacological action of a combination of an immune checkpoint inhibitor and an anticancer drug as a combination drug compared with a pharmacological action of an immune checkpoint inhibitor alone,
wherein the evaluation result is a result of evaluating the relative pharmacological action of the combination compared with a pharmacological action of an immune checkpoint inhibitor alone in a subject to be evaluated, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hKyn, hTrp, Kyn, KynA, NP, QA, and XA in blood of the subject to be evaluated, or (ii) a value of a formula in the absence of use of an anticancer drug as a combination drug and a value of the formula in the presence of the use that are calculated using the concentration value and the formula including an explanatory variable to be substituted with the concentration value and an explanatory variable on the presence or absence of the use.

17. The terminal apparatus according to claim 16, being communicatively connected via a network to an evaluating apparatus that evaluates the relative pharmacological action of the combination compared with a pharmacological action of an immune checkpoint inhibitor alone,
wherein the control unit includes:
a data transmitting unit that transmits concentration data on the concentration value or the value of the formula to the evaluating apparatus; and
the result obtaining unit receives the evaluation result transmitted from the evaluating apparatus.
